(19) United States
(12) Patent Application Publication
(10) Pub. No.: US 2023/0077896 A1
(43) Pub. Date: Mar. 16, 2023

(54) MICROBIAL LIPID PRODUCTION UTILIZING POST-FERMENTATION INDUSTRIAL WASTE STREAM FEEDSTOCKS

(71) Applicant: C16 Biosciences, Inc., New York, NY (US)

(72) Inventors: Harold M. MCNAMARA, New York, NY (US); Shara TICKU, New York, NY (US); Patrick SANFORD, New York, NY (US); Corentin MOEVUS, New York, NY (US); David HELLER, New York, NY (US)

(21) Appl. No.: 17/795,483

(22) PCT Filed: Jan. 27, 2021
(86) PCT No.: PCT/US2021/015302

§ 371 Date: Jul. 26, 2022

Related U.S. Application Data
(60) Provisional application No. 62/966,234, filed on Jan. 27, 2020.

(51) Int. Cl. C12P 7/64; C12N 1/16; C11B 13/00

(52) U.S. Cl. CPC C12P 7/64; C12N 1/16; C11B 13/00; C12N 2500/30

(57) ABSTRACT

The disclosure relates to the production of lipids by microbes utilizing novel post-fermentation industrial feedstocks. The post-fermentation industrial feedstocks comprise one or more inhibitory compounds, which traditionally have made the post-fermentation media unsuitable for utilization as a feedstock for microbial lipid production. In aspects, the disclosure provides oleaginous yeast capable of utilizing these post-fermentation industrial waste streams as a novel feedstock, methods of producing lipids and microbial oils utilizing these microbes and feedstock, and novel compositions produced from the methods.

### CROSS-REFERENCE TO RELATED APPLICATIONS

This application claims the benefit of priority to U.S. Provisional Application No. 62/966,234, filed on Jan. 27, 2020, the content of which is herein incorporated by reference in its entirety.

### FIELD OF THE DISCLOSURE

The present disclosure relates to an environmentally friendly and sustainable methodology by which oleaginous yeast are utilized to convert an otherwise discarded industrial waste stream into a valuable commodity, e.g. microbial derived palm oil alternatives.

### BACKGROUND

Palm oil is currently the most widely produced vegetable oil on the planet, as it finds uses in the manufacture of a large variety of products. It is widely used in food, as a biofuel precursor, and in soaps and cosmetics. The global demand for palm oil is approximately 57 million tons and is steadily increasing. However, the high demand for palm oil has resulted in environmentally detrimental practices related to the expansion of plantations devoted to palm oil-producing plants. Palm oil production is a leading contributor to tropical deforestation, resulting in habitat destruction, increased carbon dioxide emissions, and local smog clouds across South East Asia.

Thus, there is a need for an alternative method of producing palm oil alternatives, which does not rely upon utilization of oil palms and the associated negative environmental costs.

### BRIEF SUMMARY

The present disclosure provides an environmentally sustainable solution to traditional methods of producing palm oil. By harnessing the power of oleaginous yeast, the disclosure teaches methods and processes for producing lipid compositions (e.g. palm oil alternatives), which do not rely upon the environmentally destructive practices of harvesting oil palm plants.

Furthermore, the disclosure teaches that an otherwise discarded industrial waste stream, which contains compounds that the industry teaches are inhibitory to microbial growth, can in fact be utilized according to the taught processes, as a feedstock for oleaginous yeast to produce microbial oils.

That is, in aspects, the disclosure provides oleaginous yeast capable of growing on industrial waste stream feedstocks and converting the otherwise discarded feedstock into a valuable commodity, e.g. microbial derived palm oil alternatives.

In aspects, the industrial waste stream that is utilized as a microbial feedstock is derived from ethanol production. The industry teaches that this industrial waste stream is not suitable to utilize as a microbial feedstock, because the material contains chemical compounds and metabolites that are known to be inhibitory to microbial growth. Consequently, the material is often discarded. Alternatively, some speculated that elaborate pre-treatments and conditioning may be needed to remove detrimental and inhibitory compounds. Surprisingly, the disclosure teaches microbes, methods, and processes that utilize this material to produce lipid compounds of value, such as microbe derived palm oil alternatives.

Some in industry have utilized oleaginous yeast to catabolize a wide range of mono- and oligosaccharides found in lignocellulosic biomass, such as stover, forestry wastes, or energy crops. Lignocellulosic biomass is an attractive carbon source for bio-based fuel and chemical production, because glucose and xylose are the most abundant sugars in lignocellulosic hydrolysates, typically ranging from 60-70% glucose and 30-40% xylose, thus forming a viable source of sugar for a microbial feedstock. However, there are inefficiencies resultant from utilizing lignocellulosic hydrolysates as a feedstock for producing microbially derived palm oil alternatives.

In aspects, the present disclosure provides oleaginous microbial compositions, as described herein. In aspects, the disclosure provides methods of producing microbial lipids, as described herein. In aspects, the present disclosure provides methods of producing oleaginous microbial compositions, as described herein. In aspects, the disclosure provides methods of producing microbial lipids, as described herein.

In one aspect, the present disclosure provides an oleaginous microbial fermentation broth composition, comprising: a) a feedstock comprising at least 10 μM concentration of at least one oleaginous microbial inhibitor; b) at least 0.5 grams (g) dry cell weight (DCW) per liter (L) oleaginous microbe titer; and c) at least 0.2 g lipid per g DCW lipid content.

In some embodiments, the oleaginous microbial inhibitor is an acid.

In some embodiments, the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.

In some embodiments, the oleaginous microbial inhibitor is an aldehyde. In some embodiments, the oleaginous microbial inhibitor is 4-hydroxybenzaldehyde, furfural, or 5-hydroxymethyl-2-furaldehyde.

In some embodiments, the oleaginous microbial inhibitor is an ester. In some embodiments, the oleaginous microbial inhibitor is propamocarb.

In some embodiments, the oleaginous microbial inhibitor is a sugar alcohol. In some embodiments, the oleaginous microbial inhibitor is xylitol.

In some embodiments, the composition comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the composition comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the composition comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the composition comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the composition comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the composition comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the composition comprises at least one of the following oleaginous microbial inhibitors: 5-aminolevulinic acid, 4-hydroxybenzaldehyde, and 5-hydroxymethyl-2-furaldehyde.

In some embodiments, the composition comprises at least two or three of the following oleaginous microbial inhibitors: 5-aminolevulinic acid, 4-hydroxybenzaldehyde, and 5-hydroxymethyl-2-furaldehyde.

In some embodiments, the composition comprises each of the following oleaginous microbial inhibitors: 5-aminolevulinic acid, 4-hydroxybenzaldehyde, and 5-hydroxymethyl-2-furaldehyde.

In some embodiments, the composition comprises 4-hydroxybenzaldehyde.

In some embodiments, the composition comprises at least one of the following: at least 5 mg calcium per 100 g composition; at least 0.4 mg iron per 100 g composition; at least 100 mg potassium per 100 g composition; and at least 10 ma sodium per 100 g composition.

In some embodiments, the composition comprises each of the following: at least 5 mg calcium per 100 g composition; at least 0.4 mg iron per 100 g composition; at least 100 mg potassium per 100 g composition; and at least 10 mg sodium per 100 g composition.

In some embodiments, the oleaginous microbes are oleaginous yeast.

In some embodiments, the oleaginous microbes are oleaginous yeast of the genus Rhodosporidium, Yarrowia, or Lipomyces.

In some embodiments, the oleaginous microbes are oleaginous yeast of the genus Rhodosporidium.

In some embodiments, the oleaginous microbes are oleaginous yeast of the species Rhodosporidium toruloides, Yarrowia lipolytica, or Lipomyces starkeyi.

In some embodiments, the oleaginous microbes are oleaginous yeast of the species Rhodosporidium toruloides.

In some embodiments, the composition comprises at least 5.0 g/L DCW. In some embodiments, the composition comprises at least 10.0 g/L DCW. In some embodiments, the composition comprises at least 50.0 g/L DCW.

In some embodiments, the feedstock is a yeast fermentation waste product.

In some embodiments, the feedstock is obtained from a yeast-based bioethanol production waste stream.

In some embodiments, the feedstock is not obtained from food waste or hydrolysate from agricultural waste.

In some embodiments, the feedstock is not obtained from a lignocellulosic biomass hydrolysate.

In some embodiments, the lipid titer is at least 5 g/L. In some embodiments, the lipid titer is at least 10 g/L. In some embodiments, the lipid titer is at least 25 g/L.

In some embodiments, the composition comprises a concentration of 4-hydroxybenzaldehyde that induces a higher lipid titer compared to the composition without 4-hydroxybenzaldehyde.

In some embodiments, the lipid content is at least 0.3 g lipid/g DCW. In some embodiments, the lipid content is at least 0.5 g lipid /g DCW.

In some embodiments, the feedstock is not pre-treated. In some embodiments, the feedstock is not detoxified, hydrolyzed, or treated with activated charcoal. In some embodiments, the feedstock is not pre-treated with physical, physico-chemical, chemical, or biological means.

In some embodiments, the composition comprises a carbon source.

In some embodiments, the feedstock is a yeast fermentation waste product, and wherein the composition comprises a carbon source not originally present in the feedstock.

In some embodiments, the composition comprises a C3-C12 carbon source.

In some embodiments, the composition comprises a carbon source selected from arabinose, glucose, glycerol, sucrose, and xylose, and any combination thereof.

In some embodiments, the composition comprises a carbon source, and wherein the carbon source is glycerol.

In some embodiments, the composition comprises at least 10 g/L of a carbon source or a mixture of carbon sources. In some embodiments, the composition comprises at least 50 g/L of a carbon source or a mixture of carbon sources.

In some embodiments, the oleaginous microbes are R. toruloides, wherein the composition comprises a carbon source, and wherein the concentration of the carbon source in the composition yields a higher lipid titer from the species R. toruloides as compared to a control composition with the species Y. lipolytica or L. starkeyi.

In one aspect, the present disclosure provides an oleaginous microbial fermentation broth composition, comprising: a feedstock comprising at least 10 μM concentration of at least one oleaginous microbial inhibitor; at least 10 g/L glycerol; at least 0.5 grams (g) dry cell weight (DCW) per liter (L) oleaginous microbe titer; and at least 0.2 g lipid per g DCW lipid content.

In one aspect, the present disclosure provides a method of producing an oleaginous microbial fermentation broth composition, comprising: a) growing an oleaginous microbe on a feedstock comprising at least 10 μM concentration of at least one oleaginous microbial inhibitor, wherein said method results in a microbially produced lipid content of at least 0.2 g lipid/g DCW.

In some embodiments, the oleaginous microbial inhibitor is an acid.

In some embodiments, the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.

In some embodiments, the oleaginous microbial inhibitor is an aldehyde.

In some embodiments, the oleaginous microbial inhibitor is 4-hydroxybenzaldehyde, furfural, or 5-hydroxymethyl-2-furaldehyde.

In some embodiments, the oleaginous microbial inhibitor is an ester.

In some embodiments, the oleaginous microbial inhibitor is propamocarb.

In some embodiments, the oleaginous microbial inhibitor is a sugar alcohol.

In some embodiments, the oleaginous microbial inhibitor is xylitol.

In some embodiments, the feedstock comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least one of the following oleaginous microbial inhibitors: 5-aminolevulinic acid, 4-hydroxybenzaldehyde, and 5-hydroxymethyl-2-furaldehyde.

In some embodiments, the feedstock comprises at least two or three of the following oleaginous microbial inhibitors: 5-aminolevulinic acid, 4-hydroxybenzaldehyde, and 5-hydroxymethyl-2-furaldehyde.

In some embodiments, the feedstock comprises each of the following oleaginous microbial inhibitors: 5-aminolevulinic acid, 4-hydroxybenzaldehyde, and 5-hydroxymethyl-2-furaldehyde.

In some embodiments, the feedstock comprises 4-hydroxybenzaldehyde.

In some embodiments, the feedstock comprises at least one of the following: at least 5 mg calcium per 100 g feedstock; at least 0.4 mg iron per 100 g feedstock; at least 100 ma potassium per 100 g feedstock; and at least 10 mg sodium per 100 g feedstock.

In some embodiments, the feedstock comprises each of the following: at least 5 mg calcium per 100 g feedstock; at least 0.4 mg iron per 100 g feedstock; at least 100 mg potassium per 100 g feedstock; and at least 10 mg sodium per 100 g feedstock.

In some embodiments, the oleaginous microbes are oleaginous yeast.

In some embodiments, the oleaginous microbes are oleaginous yeast of the genus Rhodosporidium, Yarrowia, or Lipomyces.

In some embodiments, the oleaginous microbes are oleaginous yeast of the genus Rhodosporidium.

In some embodiments, the oleaginous microbes are oleaginous yeast of the species Rhodosporidium toruloides, Yarrowia lipolytica, or Lipomyces starkeyi.

In some embodiments, the oleaginous microbes are oleaginous yeast of the species Rhodosporidium toruloides.

In some embodiments, the method results in a DCW of at least 5.0 g/L.

In some embodiments, the method results in a DCW of at least 10.0 g/L.

In some embodiments, the method results in a DCW of at least 50.0 g/L.

In some embodiments, the feedstock is a yeast fermentation waste product.

In some embodiments, the feedstock is obtained from a yeast-based bioethanol production waste stream.

In some embodiments, the feedstock is not obtained from food waste or hydrolysate from agricultural waste.

In some embodiments, the feedstock is not obtained from a lignocellulosic biomass hydrolysate.

In some embodiments, the method results in a lipid titer of at least 5 g/L.

In some embodiments, the method results in a lipid titer of at least 10 g/L.

In some embodiments, the method results in a lipid titer of at least 25 g/L.

In some embodiments, the feedstock comprises a concentration of 4-hydroxybenzaldehyde that induces a higher lipid titer compared to the feedstock without 4-hydroxybenzaldehyde.

In some embodiments, the lipid content is at least 0.3 g lipid/g DCW.

In some embodiments, the lipid content is at least 0.5 g lipid/g DCW.

In some embodiments, the feedstock is not pre-treated.

In some embodiments, the feedstock is not detoxified, hydrolyzed, or treated with activated charcoal.

In some embodiments, the feedstock is not pre-treated with physical, physico-chemical, chemical, or biological means.

In some embodiments, the feedstock comprises a carbon source.

In some embodiments, the feedstock is a yeast fermentation waste product, and wherein the composition comprises a carbon source not originally present in the feedstock.

In some embodiments, the composition comprises a C3-C12 carbon source.

In some embodiments, the composition comprises a carbon source selected from arabinose, glucose, glycerol, sucrose, and xylose, and any combination thereof.

In some embodiments, the composition comprises a carbon source, and wherein the carbon source is glycerol.

In some embodiments, the composition comprises at least 10 g/L of a carbon source or a mixture of carbon sources.

In some embodiments, the composition comprises at least 50 g/L of a carbon source or a mixture of carbon sources.

In some embodiments, the oleaginous microbes are R. toruloides, wherein the composition comprises a carbon source, and wherein the concentration of the carbon source in the composition yields a higher lipid titer from the species R. toruloides as compared to a control composition with the species Y. lipolytica or L. starkeyi.

In one aspect, the present disclosure provides a method of producing microbial lipids from oleaginous microbes, comprising: a) providing a feedstock comprising at least 10 μM concentration of at least one oleaginous microbial inhibitor; and b) growing the oleaginous microbes on said feedstock, thereby producing microbial lipids.

In some embodiments, the oleaginous microbial inhibitor is an acid.

In some embodiments, the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.

In some embodiments, the oleaginous microbial inhibitor is an aldehyde.

In some embodiments, the oleaginous microbial inhibitor is 4-hydroxybenzaldehyde, furfural, or 5-hydroxymethyl-2-furaldehyde.

In some embodiments, the oleaginous microbial inhibitor is an ester.

In some embodiments, the oleaginous microbial inhibitor is propamocarb.

In some embodiments, the oleaginous microbial inhibitor is a sugar alcohol.

In some embodiments, the oleaginous microbial inhibitor is xylitol.

In some embodiments, the feedstock comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least one of the following oleaginous microbial inhibitors: 5-aminolevulinic acid, 4-hydroxybenzaldehyde, and 5-hydroxymethyl-2-furaldehyde.

In some embodiments, the feedstock comprises at least two or three of the following oleaginous microbial inhibitors: 5-aminolevulinic acid, 4-hydroxybenzaldehyde, and 5-hydroxymethyl-2-furaldehyde.

In some embodiments, the feedstock comprises each of the following oleaginous microbial inhibitors: 5-aminolevulinic acid, 4-hydroxybenzaldehyde, and 5-hydroxymethyl-2-furaldehyde.

In some embodiments, the feedstock comprises 4-hydroxybenzaldehyde.

In some embodiments, the feedstock comprises at least one of the following: at least 5 mg calcium per 100 g feedstock; at least 0.4 mg iron per 100 g feedstock; at least 100 mg potassium per 100 g feedstock; and at least 10 mg sodium per 100 g feedstock.

In some embodiments, the feedstock comprises each of the following: at least 5 mg calcium per 100 g feedstock; at least 0.4 mg iron per 100 g feedstock; at least 100 mg potassium per 100 g feedstock; and at least 10 mg sodium per 100 g feedstock.

In some embodiments, the oleaginous microbes are oleaginous yeast.

In some embodiments, the oleaginous microbes are oleaginous yeast of the genus Rhodosporidium, Yarrowia, or Lipomyces.

In some embodiments, the oleaginous microbes are oleaginous yeast of the genus Rhodosporidium.

In some embodiments, the oleaginous microbes are oleaginous yeast of the species Rhodosporidium toruloides, Yarrowia lipolytica, or Lipomyces starkeyi.

In some embodiments, the oleaginous microbes are oleaginous yeast of the species Rhodosporidium toruloides.

In some embodiments, the method results in a DCW of at least 5.0 g/L.

In some embodiments, the method results in a DCW of at least 10.0 g/L.

In some embodiments, the method results in a DCW of at least 50.0 g/L.

In some embodiments, the feedstock is a yeast fermentation waste product.

In some embodiments, the feedstock is obtained from a yeast-based bioethanol production waste stream.

In some embodiments, the feedstock is not obtained from food waste or hydrolysate from agricultural waste.

In some embodiments, the feedstock is not obtained from a lignocellulosic biomass hydrolysate.

In some embodiments, the method results in a lipid titer of at least 5 g/L.

In some embodiments, the method results in a lipid titer of at least 10 g/L.

In some embodiments, the method results in a lipid titer of at least 25 g/L.

In some embodiments, the feedstock comprises a concentration of 4-hydroxybenzaldehyde that induces a higher lipid titer compared to the feedstock without 4-hydroxybenzaldehyde.

In some embodiments, the method results in a lipid content of at least 0.3 g lipid/g DCW.

In some embodiments, the method results in a lipid content of at least 0.5 g lipid/g DCW.

In some embodiments, the feedstock is not pre-treated.

In some embodiments, the feedstock is not detoxified, hydrolyzed, or treated with activated charcoal.

In some embodiments, the feedstock is not pre-treated with physical, physico-chemical, chemical, or biological means.

In some embodiments, the feedstock comprises a carbon source.

In some embodiments, the feedstock is a yeast fermentation waste product, and wherein the composition comprises a carbon source not originally present in the feedstock.

In some embodiments, the feedstock comprises a C3-C12 carbon source.

In some embodiments, the feedstock comprises a carbon source selected from arabinose, glucose, glycerol, sucrose, and xylose, and any combination thereof.

In some embodiments, the feedstock comprises a carbon source, and wherein the carbon source is glycerol.

In some embodiments, the feedstock comprises at least 10 g/L of a carbon source or a mixture of carbon sources.

In some embodiments, the feedstock comprises at least 50 g/L of a carbon source or a mixture of carbon sources.

In some embodiments, the oleaginous microbes are R. toruloides, wherein the feedstock comprises a carbon source, and wherein the concentration of the carbon source in the feedstock yields a higher lipid titer from the species R. toruloides as compared to a control feedstock with the species Y. lipolytica or L. starkeyi.

### BRIEF DESCRIPTION OF THE SEVERAL VIEWS OF THE DRAWINGS

FIG. 1 shows an overview of methods according to the present disclosure comprising fermentation of oleaginous microbes in feedstock for the production of lipids.

FIGS. 2A-2B show an overview of exemplary sources of the oleaginous microbial feedstock of the present disclosure. FIG. 2A shows an overview of feedstocks derived from corn stillage. FIG. 2B shows an overview of different feedstock sources, including post-fermentation lignocellulosic biomass (LCB).

FIGS. 3A-3D show exemplary mass spectrometry spectra for four inhibitors identified within an exemplary feedstock of the present disclosure. FIG. 3A shows a spectrum for salicylic acid; FIG. 3B shows a spectrum for p-hydroxyphenyllactic acid; FIG. 3C shows a spectrum for 5-aminolevulinic acid; and FIG. 3D shows a spectrum for 4-hydroxybenzaldehyde. In all four spectra, the top half of the graph shows the expected reference spectrum for that inhibitor, while the bottom half shows the observed spectrum for the corresponding compound within the feedstock.

FIGS. 4A-4E show nutritional analyses for four exemplary feedstocks of the disclosure, as well as one yeast broth control. FIG. 4A shows the nutritional analysis for corn stillage syrup;

FIG. 4B shows the nutritional analysis for corn thin stillage; FIG. 4C shows the nutritional analysis for corn whole stillage; FIG. 4D shows the nutritional analysis for corn stillage pre-blend; and FIG. 4E shows the nutritional analysis for a yeast broth control.

FIG. 5 shows microbial dry cell weight (DCW) titer in grams (g) per liter (L) of culture for oleaginous yeast grown on five different feedstock conditions. From left to right: preparation A, B, C, D, E, and the control preparation.

FIG. 6 shows lipid titer in grams of lipid per liter of culture (g/L) for oleaginous yeast grown on five different feedstock conditions and a control condition. From left to right: preparation A, B, C, D, E, and the control preparation.

FIG. 7 shows lipid content in grams of lipid per grams of DCW for oleaginous yeast grown on five different feedstock conditions and a control condition. From left to right: preparation A, B, C, D, E, and the control preparation.

FIG. 8 shows the change in dry cell weight titer, lipid titer, and lipid content for the 10% supernatant feedstock condition measured at two different time points.

FIGS. 9A-9C show the results of a batch fermentation experiment for five different yeast strains, as described in Example 6, using an exemplary feedstock of the disclosure. FIG. 9A shows the DCW for these strains after fermentation; FIG. 9B shows the oil titer; and FIG. 9C shows the lipid content.

FIGS. 10A-10B show the results of a batch fermentation experiment for five different yeast strains, as described in Example 6, using an exemplary feedstock of the disclosure with added glycerol. FIG. 10A shows the DCW for these strains after fermentation, and FIG. 10B shows the oil titer.

FIGS. 11A-11E show the results of a fed-batch fermentation experiment for an exemplary strain of R. toruloides grown on two different exemplary feedstocks of the disclosure and one control defined media, each of which feedstocks were periodically fed with glycerol. FIG. 11A shows the DCW results after fermentation; FIG. 11B shows the oil titer; FIG. 11C shows the lipid content; FIG. 11D shows the productivity; and FIG. 11E shows the yield.

FIG. 12 shows the DCW results for fermentation of two exemplary strains of R. toruloides on three different exemplary feedstocks of the disclosure.

FIG. 13 shows the carbon source consumption over time of five different carbon sources by exemplary R. for toruloides strain A, as measured via HPLC.

FIGS. 14A-14B show the results of an inhibitor (4-hydroxybenzaldehyde) titration experiment on two different yeast strains, using a defined rich media +inhibitor as the feedstock and defined rich media alone (“0”) as the control. FIG. 14A shows the DCW for these strains after fermentation, and FIG. 14B shows the oil titer.

### DETAILED DESCRIPTION

The present disclosure relates to novel feedstocks, methods of using these feedstocks, oleaginous yeast capable of being utilized in the taught methods and converting the feedstocks into microbial lipids and valuable products of interest.

### DEFINITIONS

As used herein, the singular forms “a,” “an,” and “the” include plural referents unless the content clearly dictates otherwise.

“Bioreactor” and “fermentor” mean an enclosure or partial enclosure, such as a fermentation tank or vessel, in which oleaginous microorganisms are cultured typically in suspension.

“Cultivate,” “culture,” and “ferment”, and variants thereof, refer to the intentional fostering of growth and/or propagation of one or more cells, typically oleaginous yeast, by use of culture conditions. Intended conditions exclude the growth and/or propagation of microorganisms in nature (without direct human intervention).

“Dry cell weight” and “dry cell mass” as used herein refers to the weight or (mass) of the microorganism cells when collected and separated from an aqueous culture medium. It is determined in the relative absence of water. For example, reference to oleaginous yeast biomass as comprising a specified percentage of lipid by dry cell weight means that the percentage of lipid is calculated based on the weight of lipid in the biomass after substantially all water has been removed.

“Chemical composition” as used herein refers to the set of component molecular species in an oil sample, as well the relative proportion that each molecular species contributes to total sample mass.

“Continuous culture” as used herein refers to a long-term culture (greater than 1 week in which the organisms are maintained in a particular growth phase (either log or stationary).

“Culture medium” a solid, liquid, or semi-solid designed to support the growth of microorganisms or cells.

“Fatty acid profile” as used herein refers to how specific fatty acids contribute to the chemical composition of an oil titer.

“Growth” means an increase in cell size, total cellular contents, and/or cell mass or weight of an individual cell, including increases in cell weight due to conversion of a fixed carbon source into intracellular oil.

“Heterotrophic cultivation,” “heterotrophic culture,” “heterotrophic fermentation,” and variants thereof refer to the intentional fostering of growth (increases in cell size, cellular contents, and/or cellular activity) of an oleaginous microorganism in the presence of a fixed carbon source. Heterotrophic cultivation can be performed in the absence of light. Cultivation in the absence of light means cultivation of microbial cells in the complete absence or near complete absence of light where the cells do not derive a meaningful amount of their energy from light (i.e., less than 0.1%).

“Increased lipid yield” means an increase in the lipid/oil productivity of a microbial culture that can achieved by, for example, increasing the dry weight of cells per liter of culture, increasing the percentage of cells that contain lipid, and/or increasing the overall amount of lipid per liter of culture volume per unit time.

“Lignocellulosic biomass” or “LCB” refers to plant biomass that is composed of cellulose, hemicellulose, and lignin. Sources of LCB include, for example, sugar cane bagasse, sugar beet pulp, corn stover, wood chips, sawdust, and switchgrass.

“Corn stover,” a common source of LCB, consists of the leaves, stalks, and cobs of maize (Zea mays ssp. mays L.). Such stover makes up about half of the yield of a corn crop and is similar to straw from other cereal grasses. Corn stover is a very common agricultural product in areas of large amounts of corn production. Thus, corn stover contains the “non-grain” part of harvested corn. The grain or “kernel” of corn is not part of corn stover and is not a source of LCB. Rather, corn kernels or grain are often utilized for ethanol production by either dry milling or wet milling processes.

“Lipid” means any of a class of molecules that are soluble in nonpolar solvents (such as ether and hexane) and relatively or completely insoluble in water. Lipid molecules have these properties, because they are largely composed of long hydrocarbon tails that are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); and nonglycerides (sphingolipids, tocopherols, tocotrienols, sterol lipids including cholesterol and steroid hormones, prenol lipids including terpenoids, fatty alcohols, waxes, and polyketides).

“Microorganism” and “microbe” mean any microscopic unicellular organism and can include bacteria, algae, yeast, or fungi.

“Oleaginous” as used herein refers to material, e.g., a microorganism, which contains a significant component of oils, or which is itself substantial composed of oil. An oleaginous microorganism can be one that is naturally occurring or synthetically engineered to generate a significant proportion of oil.

“Oleaginous yeast” as used herein refers to a collection of yeast species that can accumulate a high proportion of their biomass as lipids (namely greater than 20% of dry cell mass). An oleaginous yeast can be one that is naturally occurring or synthetically engineered to generate a significant proportion of oil.

“Pre-treatment” refers to the physical, mechanical, and biological means employed to break down the cellulose, hemicellulose, and lignin comprised by lignocellulosic biomass (LCB) in order to release the sugars and nutrients comprised by the LCB for consumption by microorganisms.

“Rhodosporidium toruloides” refers to a particular species of oleaginous yeast. Previously called Rhodotorula glutinis or Rhodotorula gracilis. Also abbreviated as R. toruloides. This species includes multiple strains with minor genetic variation.

For the purposes of this disclosure, “single cell oils,” “SCOs,” and “oils” refer to microbial lipids produced by oleaginous microorganisms.

“Tailored fatty acid profile” as used herein refers to a fatty acid profile in an oil which has been manipulate towards target properties, either by changing culture conditions, the specicies of heterotrophic yeast, or by genetically modifying the heterotrophic yeast.

“Titer” as used herein refers to both a specific product of a bioprocess, as well as the amount of product produced by the bioprocess. In the present examples, titers refer to both the specific oil produced, as well as the amount and yield of oil produced.

“Transformation” as used herein refers to direct altering of the genetic material of a cell or organism through the uptake of exogenous genetic material. Example methods include electroporation, transformation, or vector-mediated transformation (for example, agrobacterium mediated transformation, or AMT).

“W/W” or “w/w”, in reference to proportions by weight, refers to the ratio of the weight of one substance in a composition to the weight of the composition. For example, reference to a composition that comprises 5% w/w oleaginous yeast biomass means that 5% of the composition's weight is composed of oleaginous yeast biomass (e.g., such a composition having a weight of 100 mg would contain 5 mg of oleaginous yeast biomass) and the remainder of the weight of the composition (e.g., 95 mg in the example) is composed of other ingredients.

“Yield” as used herein refers to the amount of product produced by a bioprocess culture, here measured in units of density (i.e., mass of product per unit culture volume).

### FEEDSTOCK

The present disclosure relates to novel feedstocks for the growth and fermentation of oleaginous microorganisms.

### Sources

In some embodiments, the feedstock is obtained from an industrial waste stream. In some embodiments, the feedstock is obtained from a bioethanol production waste stream. In some embodiments, the feedstock is obtained from a yeast fermentation waste stream. In some embodiments, the feedstock is obtained from a yeast ethanol fermentation waste stream. In some embodiments, the feedstock is obtained from corn stillage. In some embodiments, the feedstock is obtained from a biofuel or biodiesel waste stream.

In some embodiments, the feedstock is obtained from Brewers' spent yeast. In some embodiments, the feedstock is obtained from biomass waste generated by a fermentation process. In some embodiments, the feedstock is obtained from a post-fermentation lignocellulosic biomass waste stream.

In some embodiments, the source of the feedstock is a post-fermentation industrial waste stream that provides a low cost, reliable source for the growth and fermentation of the oleaginous microorganisms according to the present disclosure.

In some embodiments, the feedstock is not obtained from food waste. In some embodiments, the feedstock is not obtained from municipal wastewater.

In some embodiments, the feedstock is not obtained from hydrolysate from agricultural waste. In some embodiments, the feedstock may be obtained from a post-fermentation industrial waste stream that originates from agricultural waste.

In some embodiments, the feedstock is not obtained from lignocellulosic biomass (“LCB”). In some embodiments, the feedstock may be obtained from post-fermentation LCB.

In some embodiments, the feedstock is not yeast extract. While the feedstock may comprise some quantity of yeast content, it differs from yeast extract which is obtained by obtaining yeast cells, heating them until they rupture, then allowing the cells' own digestive enzymes to break their proteins down into simpler compounds (amino acids and peptides) through a process called autolysis. The insoluble cell walls are then separated by centrifuge, and the remaining solution is filtered, concentrated, and usually spray dried.

### Bioethanol Industrial Waste Stream

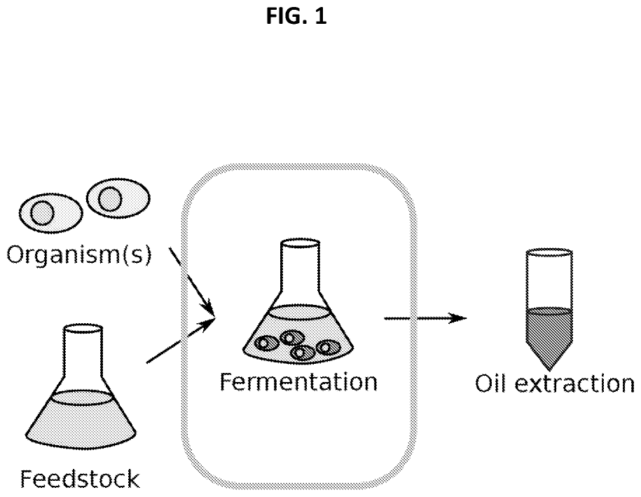

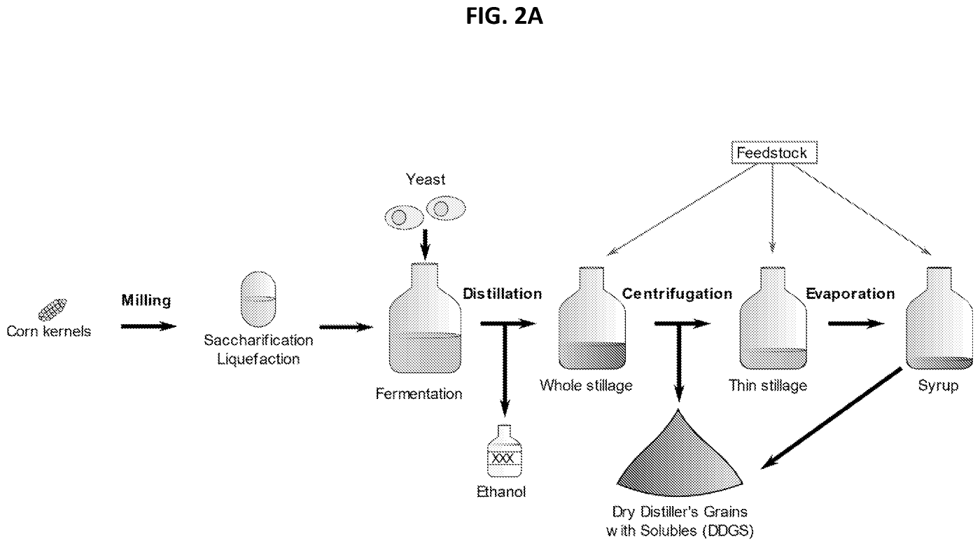

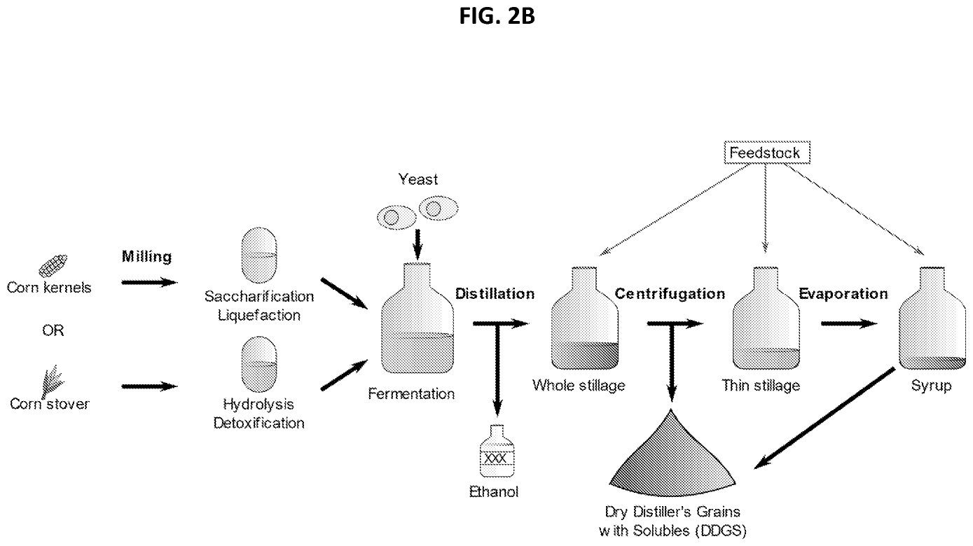

The present disclosure provides feedstocks obtained from a bioethanol industrial waste stream, compositions comprising, and methods of use thereof, e.g., for the production of oil as in FIG. 1. FIG. 2 depicts some of the sources of the feedstock of the present disclosure. In the production of bioethanol, corn kernels may be employed, as depicted in FIG. 2A. Traditionally, ethanol from corn has primarily been produced through dry- and wet-milling processes. The traditional dry-grind process involves grinding the whole corn kernel and mixing it with water and enzymes to produce a mash. The mash is cooked to liquefy the starch further, cooled, and mixed with more enzymes to convert the remaining sugar polymers to glucose before fermenting to ethanol. Wet milling involves steeping the corn for up to 48 hours to assist in separating the parts of the corn kernel. Processing the slurry separates the germ from the rest of the kernel, which is processed further to separate the fiber, starch, and gluten. The fiber and corn gluten become components of animal feed while the starch is fermented to become ethanol, corn starch, or corn syrup.

In the case of dry milling, after the corn is subjected to milling, saccharification, liquefaction, and fermentation with yeast to produce ethanol, ethanol is extracted. After ethanol extraction, the fermentation waste product is referred to as whole stillage. The solids in the whole stillage may be predominantly separated from the liquid, which liquid is then referred to as thin stillage. This thin stillage may then be evaporated to form syrup. The feedstock of the present disclosure may be derived from whole stillage, thin stillage, clarified stillage, syrup, or any combination thereof.

The feedstock of the present disclosure may also be derived from other post-fermentation waste streams, such as post-fermentation LCB feedstocks. In some embodiments, the post-fermentation industrial waste product for use in the feedstock of the present disclosure may comprise post-fermentation waste products from corn kernel-based fermentation processes, LCB fermentation processes, or a combination thereof, as depicted in FIG. 2B.

### Lignocellulosic Biomass and Feedstocks Derived Therefrom

The industrial waste stream product for use as a feedstock of the present disclosure is not itself lignocellulosic biomass (LCB). LCB refers to vegetal dry matter biomass. It may be obtained from forest residues such as wood, sawdust, wood chips, barks, dead branches; agricultural residues such as rice straw, wheat straw, cane bagasse, corn cob, and corn stover (but not corn grain or kernels); industrial residues such as pulp and paper processing waste; energy crops such as switchgrass; marine biomass; and municipal solid waste, such as food waste and waste paper. It is composed of carbohydrate polymers (cellulose, hemicellulose), and an aromatic polymer (lignin). Though LCB is plentiful, the complex structure of LCB, mainly composed of cellulose, hemicellulose, and lignin, makes it challenging to be depolymerized. In addition, wide variation in composition and contamination makes LCB difficult to employ. Its characteristics and use in, e.g., bioethanol production are covered extensively in Abo et al., Rev. Environ. Health 2019; 34(1): 57-68, herein incorporated by reference in its entirety.

Agricultural waste may include the by-products or residues of agro-forestry and wood industry processes. This may include products obtained from forest and agricultural harvest, sawdust, tipping, burdocks, and fragments from pulping and sawing.

Food waste contains approximately 47% of carbon in the dry matter, and the carbohydrate content in packed food is approximately 67%. See Zhang et al, “Characterization of food waste as feedstock for anaerobic digestion” Bioresource Technology 2007; 98(4): 929-935 and Davis “Parameter estimation for simultaneous saccharification and fermentation of food waste into ethanol using Matlab Simulink” Applied Biochemistry and Biotechnology, 2008; 147(1-3): 11-21.

The exact composition of LCB varies from source to source depending on the plant species, growth conditions and types of plant tissue, but usually comprises 35-50% cellulose, 20-35% hemicellulose and 10-25% lignin.

Cellulose is a linear glucose homopolymer with a molecular weight from 50 kDa to 2.5 10⁶ kDa, or between 300 and 15,000 glucose residues, consisting of repeated cellobiose units (two glucoses bound by a β-1, 4-oxidic bond) that form crystalline structures resistant to hydrolysis.

Hemicellulose has a structural role in lignocellulosic plants, allowing for the bonding between cellulose and lignin. Unlike cellulose, hemicellulose forms a family of various heteropolysaccharide compounds. The monomeric units of these carbohydrate macromolecules are pentose (mainly xylose and arabinose), hexose (mainly glucose, mannose, galactose) and carboxylic acids (mainly mannuronic acids and galacturonic acids). Xylan, a homopolymer consisting of xylose, is the major constituent of the hemicellulose found in hardwoods, whereas mannose is the most prevalent constituent of softwoods, predominantly found as O-acetyl-galactoglucomannan. Compared with cellulose, hemicelluloses exhibit smaller amorphous chains, which facilitates their hydrolysis.

Lignin is a cross-linked polymer with molecular masses in excess of 10 kDa. It is relatively hydrophobic and rich in aromatic subunits. The degree of polymerisation is difficult to measure, since the material is heterogeneous. Lignin is polymerized from three phenylpropanoid monomers, the alcohols P-coumaryl, coniferyl and sinapyl, which are also known as monolignols H, G and S, respectively. The ratio of lignin in plants varies widely: softwood contains 25-35% by mass, hardwood contains 20-25%, and agricultural residues contain 5-15%.

In some embodiments, the disclosed feedstocks are obtained from an industrial waste stream comprising less than 35% cellulose, 20% hemicellulose, and/or 10% lignin. In some embodiments, the disclosed feedstocks are obtained from an industrial waste stream comprising virtually no cellulose, hemicellulose, and/or lignin.

Pre-treatment of LCB is carried out to release the sugar from the crystalline lignocellulosic macrofibers and microfibers prior to hydrolysis. However, the pretreatment process is complicated by the fact that degradation of the sugars in LCB leads to the formation of fermentation inhibitors. Pre-treatment accounts for more than 20% of the total cost of manufacturing bioethanol, and is essential because it increases the hydrolysis yield by 20% without pre-treatment to more than 90% with pre-treatment. In contrast to the industrial fermentation waste stream feedstock according to the present disclosure, LCB must be pre-treated and delignified prior to use as a microbial feedstock, in order to release the sugars that act as the food source for the microorganisms. LCB may be pre-treated via physical means, such as mechanical comminution or ultrasound; physico-chemical means such as hydrothermal processes or steam explosion treatment; chemical means, such as hydrogen peroxide, concentrated acid, dilute acid, alkali, sodium chlorite, or ammonia fiber expansion treatment; and biological means, such as microbial enzyme treatment. The cellulose and hemicellulose in LCB must also be hydrolyzed either enzymatically or chemically into sugars prior to use as a feedstock. These processes may produce numerous toxic byproducts unsuitable for microbial growth, which must then be removed prior to use as a feedstock. For example, after pre-treatment and hydrolysis, LCB feedstocks often comprise inhibitory concentrations of the inhibitors furfural and/or hydroxymethylfurfural. The numerous pre-treatment requirements for LCB-based feedstocks increase the cost of oleaginous microbial fermentation and the cost of the resulting lipids. Additional pre-treatment methods for LCB may be found in Saini et al., “Lignocellulosic agriculture wastes as biomass feedstocks for second-generation bioethanol production: concepts and recent developments” 3 Biotech 2015 Aug; 5(4):337-353, herein incorporated by reference.

In some embodiments, the waste stream used for the feedstock of the present disclosure does not require pre-treatment, hydrolysis, or inhibitor removal. In some embodiments, the waste stream used for the feedstock of the present disclosure comprises furfural and/or hydroxymethylfurfural but does not require inhibitor removal to be used as a feedstock for an oleaginous microorganism, e.g., R. toruloides.

### Preparation for Use as Feedstock

Prior to use as a feedstock in culturing oleaginous microorganisms, the industrial fermentation waste stream product of the present disclosure may be sterilized, filtered, diluted, and/or centrifuged. In some embodiments, filtration removes particulates above a certain molecular weight cut off

In some embodiments, the industrial fermentation waste product may be diluted prior to use as a feedstock. In some embodiments, dilution is performed with water. In some embodiments, dilution is performed with a solvent. The degree of dilution may depend on the type of waste product employed. For example, in the case of syrup derived from corn stillage, this product is highly concentrated and viscous. For use as a feedstock, corn stillage syrup may therefore be diluted 50-95% to improve viscosity and/or aeration for the oleaginous microorganisms. Dilution may be calibrated to optimize growth parameters. In the case of whole stillage or thin stillage, which have higher moisture contents than syrup, less dilution may be required to optimize growing conditions. In some embodiments, the feedstock is diluted about 5%, 10%, 15%, 20%, 25%, 30%, 35%, 40%, 45%, 50%, 55%, 60%, 65%, 70%, 75%, 80%, 85%, 90%, or 95%, or any ranges or subranges therebetween.

In some embodiments, the industrial fermentation waste product is sterilized prior to use as a feedstock. In some embodiments, sterilization comprises autoclaving. In some embodiments, sterilization is carried out through the use of heat. For example, the feedstock may be exposed to temperatures above 100° C., e.g., 115-125° C., for 20-40 minutes. In some embodiments, the feedstock is treated with high-pressure steam sterilization. In some embodiments, sterilization comprises cool sterilization through the use of, e.g., hydrogen peroxide and catalase. In some embodiments, the feedstock may be sterilized by filtration.

In some embodiments, the waste stream product is centrifuged and only the supernatant is employed within the feedstock. In some embodiments, the entire waste stream product is employed within the feedstock, without centrifugation or separation.

In some embodiments, the feedstock may be supplemented with trace minerals and/or additional nutrient sources to encourage additional growth. In some embodiments, the feedstock is employed without supplementation.

In some embodiments, the feedstock according to the present disclosure does not require pre-treatment. In some embodiments, the industrial fermentation waste stream product does not need to be pre-conditioned with any of the methods employed for the pre-treatment of LCB prior to use for the fermentation of oleaginous microorganisms. In some embodiments, the industrial fermentation waste stream product may be a post-fermentation LCB waste stream, which does not require the pre-treatment of LCB prior to use.

In the case of feedstocks derived from food waste, prior to its use as a feedstock, food waste must be ground (i.e., pulverized), mixed with significant additional liquid, and then hydrolyzed, e.g., via addition of sulfuric acid and neutralization with sodium hydroxide. In some embodiments, the post-fermentation industrial waste stream used for the feedstock of the present disclosure does not require pulverization, hydrolysis, or neutralization prior to use as a feedstock.

### Molecular Signature

In some embodiments, the feedstock is the waste product from yeast fermentation, such that its nutrient content is lower than the nutrient content in feedstock obtained from other sources, such as LCB, food waste or agricultural waste.

In some embodiments, the feedstock differs from feedstock derived from food waste. After pre-treatment, feedstock derived from food waste has greater than 90% of the sugar content composed of glucose. Sugar concentration is approximately 50 g/L; nitrogen concentration is around 4 g/L; and phosphorous concentration is around 1.5 g/L. Alternatively, depending on the amount of water used in pre-treatment, these values may differ but be maintained in a comparable ratio. See Chi et al., “Lipid Production by Culturing Oleaginous Yeast and Algae with Food Waste and Municipal Wastewater in an Integrated Process” Applied Biochemistry and Biotechnology 2011; 165(2): 442-453.

In some embodiments, the feedstock differs from yeast extract. The composition of yeast extract is roughly 50-75% protein content, 8-12% nitrogen and 4-13% carbohydrate content, with little to no lipid content.

In some embodiments, the feedstock differs from LCB feedstocks. Feedstocks derived from LCB contain significant quantities of glucose derived from the cellulose comprised by the LCB. The exact quantity depends on the source of the LCB and the efficiency of the pre-treatment methods employed. For example, glucose concentrations in feedstock obtained from corn stover may reach over 40 g/L and concentrations of xylose in corn stover can be around 10 g/L (see, e.g., Li et al., Bioresource Technology 2018; 269: 400-407). In some embodiments, the feedstock may be obtained from a post-fermentation LCB feedstock.

In some embodiments, the feedstocks according to the present disclosure are obtained from corn stillage. Corn DDGS or stillage is a distillation by-product of ethanol production. Stillages (vinasse) following distillation of ethanol from industrial ethanol fermentations of grain comprise numerous ingredients, including corn gluten, yeast protein, residual corn fiber, yeast cells, corn oil, and dissolved organics. Thin stillage contains significant quantities of glycerol (14 to 20 g/L), glucose disaccharides (e.g., cellobiose, trehalose, etc.) (6 to 10 g/L), xylose, lactic acid, corn oil and various oligosaccharides derived from residual undigested starch, dextrins, cellulose and hemicellulose. The total dissolved and suspended organic content of thin stillage is about 10% w/v. In contrast to the composition of LCB-derived feedstocks or food waste-derived feedstocks, thin stillage comprises very little glucose—only around 0.9 g/L. The concentration of other components are around the following values: glucan (oligosaccharide) 12.4 g/L; Xylose 0.7 g/L; Xylan (oligosaccharide) 3.7 g/L; Arabinose 0.4 g/L; Arabinan (oligosaccharide) 0.5 g/L; Lactic acid 16.8 g/L; Glycerol 14.4 g/L; Acetic acid 0.3 g/L; Butanediol 1.9 g/L; Ethanol 0.6 g/L. See Kim et al., “Composition of corn dry-grind ethanol by-products: DDGS, wet cake, and thin stillage” Bioresour Technol 2008;99(12):5165-76. Stillage can also have relatively high nitrogen and phosphorous contents, about 2 g/L and 130 mg/L, respectively (Yen et al. 2012).

In some embodiments, the feedstock may be characterized by the concentration of glycerol. In some embodiments, the feedstock comprises at least 10, 15, 20, 25, 30, 35, 40, 45, 50, 55, 60, 65, 70, 75, 80, 85, 90, 95, or 100 g/L glycerol. In some embodiments, the feedstock comprises at least 10 g/L glycerol. In some embodiments, the feedstock comprises at least 50 g/L glycerol. In some embodiments, the feedstock comprises at least 70 g/L glycerol.

In some embodiments, the feedstock may be characterized by a relative absence of glucose. In some embodiments, the glucose content of the feedstock is less than 10, 9, 8, 7, 6, 5, 4, 3, 2, or 1 g/L. In some embodiments, the glucose content of the feedstock is less than 5 g/L. In some embodiments, the glucose content of the feedstock is less than 2 g/L. In some embodiments, the glucose content of the feedstock is less than 1 g/L.

In some embodiments, the feedstock may be characterized by a relative absence of xylose. In some embodiments, the xylose content of the feedstock is less than 10, 9, 8, 7, 6, 5, 4, 3, 2, or 1 g/L. In some embodiments, the xylose content of the feedstock is less than 5 g/L. In some embodiments, the xylose content of the feedstock is less than 2 g/L. In some embodiments, the xylose content of the feedstock is less than 1 g/L.

In some embodiments, the feedstock may comprise various amounts of the grain ethanol distillation stillage or processed grain ethanol distillation stillage. In some embodiments, the medium may comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 99%, or about 100%, or any ranges or subranges therebetween, of grain ethanol distillation stillage or processed grain ethanol distillation stillage. The grain ethanol distillation stillage or processed grain ethanol distillation stillage may be diluted with water or other solvents. For example, in some embodiments, where the industrial post-fermentation waste product is thin stillage, the feedstock may comprise 50-100% of the thin stillage. In some embodiments, where the industrial post-fermentation waste product is syrup, the feedstock may comprise 10-50% of the syrup.

In some embodiments, the feedstock comprises a higher concentration of trace metals, minerals, and/or salts than a control feedstock. In some embodiments, the control feedstock is a defined medium, e.g., yeast extract-peptone-dextrose (YPD) medium, which is generally composed of 10 g/L yeast extract, 20 g/L peptone, and 20 g/L dextrose. In some embodiments, the feedstock comprise higher concentrations of any one of the following elements, as compared to a defined medium: calcium, iron, potassium, and sodium. In some embodiments, the feedstock comprise higher concentrations of each of the following elements, as compared to a defined medium: calcium, iron, potassium, and sodium.

In some embodiments, the feedstock comprises at least 5, 10, 15, 20, 25, 30, 35, 40, 45, 50, 55, 60, 65, 70, 75, 80, 85, 90, 95, 100, 150, 200, 250, 300, 350, 400, 450, or 500 mg calcium/100 g feedstock, or any ranges or subranges therebetween. In some embodiments, the feedstock comprises at least 5 mg calcium/100 g feedstock. In some embodiments, the feedstock comprises about 5-100 mg calcium/100 g feedstock.

In some embodiments, the feedstock comprises at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg iron/100 g feedstock, or any ranges or subranges therebetween. In some embodiments, the feedstock comprises at least 0.1 mg iron/100 g feedstock. In some embodiments, the feedstock comprises about 0.1-10 mg iron/100 g feedstock.

In some embodiments, the feedstock comprises at least 100, 150, 200, 250, 300, 350, 400, 450, 500, 550, 600, 650, 700, 750, 800, 850, 900, 1000, 1100, 1200, 1300, 1400, 1500, 1600, 1700, 1800, 1900, 2000, 2100, 2200, 2300, 2400, 2500, 2600, 2700, 2800, 2900, or 3000 mg potassium/100 g feedstock, or any ranges or subranges therebetween. In some embodiments, the feedstock comprises at least 100 mg potassium/100 g feedstock. In some embodiments, the feedstock comprises about 100-2000 mg potassium/100 g feedstock.

In some embodiments, the feedstock comprises at least 10, 15, 20, 25, 30, 35, 40, 45, 50, 55, 60, 65, 70, 75, 80, 85, 90, 95, 100, 150, 200, 250, 300, 350, 400, 450, or 500 mg sodium/100 g feedstock, or any ranges or subranges therebetween. In some embodiments, the feedstock comprises at least 10 mg sodium/100 g feedstock. In some embodiments, the feedstock comprises about 10-200 mg sodium/100 g feedstock.

### Inhibitors

In some embodiments, the feedstock comprises one or more microorganism inhibitors at a concentration of greater than or equal to 10 μM. In some embodiments, the feedstock comprises an inhibitor that is an acid. In some embodiments, the acid is 5-aminolevulinic acid, mevalonic acid, pyroglutamic acid, p-hydroxyphenyllactic acid, salicylic acid, alpha-hydroxyisocaproic acid, succinic acid-2,2,3,3-d4, or citric acid. In some embodiments, the feedstock comprises an inhibitor that is an aldehyde. In some embodiments, the aldehyde is 4-hydroxybenzaldehyde, furfural, or 5-hydroxymethyl-2-furaldehyde. In some embodiments, the feedstock comprises an inhibitor that is an ester. In some embodiments, the ester is propamocarb. In some embodiments, the feedstock comprises an inhibitor that is a sugar alcohol. In some embodiments, the sugar alcohol is xylitol.

In some embodiments, the feedstock comprises at least one of the following inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol. In some embodiments, the feedstock comprises each of the following inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least one of the following inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol. In some embodiments, the feedstock comprises each of the following inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

In some embodiments, the feedstock comprises at least one of the following inhibitors: 5-aminolevulinic acid, 5-hydroxymethyl-2-furaldehyde, and 4-hydroxybenzaldehyde. In some embodiments, the feedstock comprises each of the following inhibitors: 5-aminolevulinic acid, 5-hydroxymethyl-2-furaldehyde, and 4-hydroxybenzaldehyde.

In some embodiments, the feedstock comprises 5-aminolevulinic acid. 5-Aminolevulinic acid (also referred to as δ-Aminolevulinic acid, dALA, δ-ALA, and 5ALA) is an endogenous non-proteinogenic amino acid and is the first compound in the porphyrin synthesis pathway that leads to heme in mammals and chlorophyll in plants. It is also produced by fungi, protozoa, and bacteria. In non-photosynthetic eukaryotes, 5-aminolevulinic acid is produced by the enzyme ALA synthase, from glycine and succinyl-CoA. This reaction is known as the Shemin pathway, which occurs in mitochondria. 5-aminolevulinic acid is known to inhibit the growth of microorganisms. See, e.g., “Antifungal effect of 5-aminolevulinic acid PDT in Trichophyton rubrum,” Kamp et al., Mycoses 2005;48(2):101-7, incorporated herein by reference, showing the inhibition of Trichophyton rubrum with 5′aminolevulinic acid at concentrations above 10 mM. In some embodiments, the feedstock comprises 5′aminolevulinic acid. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM 5′aminolevulinic acid, or any ranges or subranges therebetween. In some embodiments, the feedstock comprises at least 10 μM 5′aminolevulinic acid. In some embodiments, the feedstock comprises about 10-200 μM 5′aminolevulinic acid.

In some embodiments, the feedstock comprises mevalonic acid lactone. Other names for this inhibitor include (±)-Mevalonolactone; dl-Mevalonic acid lactone; 4-Hydroxy-4-methyltetrahydro-2H-pyran-2-one; tetrahydro-4-hydroxy-4-methyl-2H-Pyran-2-one; (RS)-Mevalonolactone; Mevalolactone; Mevalonic acid lactone; β-Hydroxy-β-methyl-δ-valerolactone; MVSL; Mevalonic lactone; Mevalonic acid δ-lactone; (±)-tetrahydro-4-hydroxy-4-methyl-2H-pyran-2-one. Alkaline hydrolysis of mevalonic acid lactone produces mevalonate, a precursor of farnesyl and geranylgeranyl pyrophosphates which are required for protein prenylation. Mevalonic acid lactone exists in equilibrium with mevalonic acid, such that the two terms may be used interchangeably for the purposes of the present disclosure. Mevalonic acid is a precursor in the biosynthetic pathway known as the mevalonate pathway that produces terpenes and steroids. Mevalonic acid is the primary precursor of isopentenyl pyrophosphate (IPP) that is in turn the basis for all terpenoids. At concentrations in the tens of micromolar (e.g., 67 μM), mevalonic acid lactone can act as an inhibitor for microorganism growth. For example, the publication “New types of antimicrobial compounds produced by Lactobacillus plantarum,” Niku-Paavola et al., J. Applied Microbiol. 1999;86(1):29-35, herein incorporated by reference, shows that inhibitors such as mevalonic acid lactone inhibited the growth of a test organism, Pantoea agglomerans, by 10-15% at concentrations of just 10 ppm, but this level rose to 100% inhibition when the inhibitors were combined. Such synergistic effects among microbial inhibitors would be expected in view of these findings and similar results known in the art. Mevalonic acid lactone was also observed to inhibit the growth of the fungus Fusarium avenaceum. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM mevalonic acid lactone, or any ranges or subranges therebetween. In some embodiments, the feedstock comprises at least 10 μM mevalonic acid lactone.

In some embodiments, the feedstock comprises pyroglutamic acid. Pyroglutamic acid (also known as PCA, 5-oxoproline, pidolic acid) is a natural amino acid derivative in which the free amino group of glutamic acid or glutamine cyclizes to form a lactam. It is a metabolite in the glutathione cycle that is converted to glutamate by 5-oxoprolinase. N-terminal glutamic acid and glutamine residues can spontaneously cyclize to become pyroglutamate, or be enzymatically converted by glutaminyl cyclases. Pyroglutamic acid is a known inhibitor of fungal species, such as Pseudoperonospora cubensis. See, e.g., “Bioassay-Guided Isolation of Antifungal Compounds from Disporopsis aspersa against Pseudoperonospora cubensis and Phytophthora infestans,” Zhu et al., Chemistry & Biodiversity 2018;15(7):e1800090, incorporated herein by reference. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM pyroglutamic acid, or any ranges or subranges therebetween. In some embodiments, the feedstock comprises at least 10 μM pyroglutamic acid.

In some embodiments, the feedstock comprises p-hydroxyphenyllactic acid. p-Hydroxyphenyllactic acid may also be referred to as 2-Hydroxy-3-(4-hydroxyphenyl)propanoate; 2-Hydroxy-3-(p-hydroxyphenyl)propionic acid; 4-Hydroxyphenyllactic acid; beta-(4-Hydroxyphenyl)lactic acid; beta-(p-Hydroxyphenyl)lactic acid; DL-p-Hydroxyphenyllactic acid; Hydroxyphenyllactic acid; or 4-hydroxyphenyllactate (the L-foim). p-Hydroxyphenyllactic acid is a tyrosine metabolite. It belongs to the class of organic compounds known as phenylpropanoic acids. Phenylpropanoic acids are compounds with a structure containing a benzene ring conjugated to a propanoic acid. Hydroxyphenyllactic acid is a microbial metabolite found in Acinetobacter, Bacteroides, Bifidobacteria, Bifidobacterium, Clostridiurn, Enterococcus, Escherichia, Eubacteriurn, Klebsiella, Lactobacillus, Pseudomonas and Staphylococcus. Hydroxyphenyllactic acid is a known inhibitor of microorganisms, such as Endomyces fibuliger and Aspergillus fumigatus. See Lavermicocca et al., “Purification and characterization of novel antifungal compounds from the sourdough Lactobacillus plantarum strain 21B” Appl Environ Microbiol 2000 Sep;66(9):4084-90 and Ryan et al., “Lactobacillus amvlovorus DSM 19280 as a novel food-grade antifungal agent for bakery products” Int J Food Microbiol 2011:146(3):276-83 (showing inhibitory concentrations of e.g., 5 mg/mL), each of which is incorporated herein by reference. In some embodiments, the feedstock comprises at least 10 μM, 20 μM, 30 μM, 40 μM, 50 μM, 60 μM, 70 μM, 80 μM, 90 μM, 100 μ, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM p-hydroxyphenyllactic acid, or any ranges or subranges therebetween. In some embodiments, the feedstock comprises at least 10 μM p-hydroxyphenyllactic acid.

In some embodiments, the feedstock comprises salicylic acid. This compound may also be referred to as 2-Hydroxybenzoic acid, o-hydroxybenzoic acid, or 2-Carboxyphenol. Salicylic acid is a lipophilic monohydroxybenzoic acid, a type of phenolic acid, and a beta hydroxy acid (BHA). It is biosynthesized from the amino acid phenylalanine. Salicylic acid is commonly used as a food preservative, a bactericidal, and an antiseptic. It also has a role as an antiinfective agent, an antifungal agent, a keratolytic drug, an EC 1.11.1.11 (L-ascorbate peroxidase) inhibitor, a plant metabolite, an algal metabolite and a plant hormone. It has known anti-microbial activity against bacteria and fungi generally. See, e.g., Amborabé et al., “Antifungal effects of salicylic acid and other benzoic acid derivatives towards Eutypa lata: structure-activity relationship” Plant Physiol and Biochem 2002; 40(12):1051-60 (herein incorporated by reference) which shows that even concentrations as low as 1 mM caused fungistatic effects in Eutypa lata and lowered growth rate even after renewal of the culture medium. See also Ryan et al., “Lactobacillus amvlovorus DSM 19280 as a novel food-grade antifungal agent for bakery products” Int J Food Microbiol 2011;146(3):276-83 (herein incorporated by reference) showing a 0.25 mg/mL inhibitory concentration against Aspergillus fumigatus. In addition, Scharff et al., “The Effects of Salicylic Acid on Metabolism and Potassium Ion Content in Yeast” Experimental Biology and Medicine 1976;151(1):72-7 (herein incorporated by reference) shows the fungicidal effects of salicylic acid against Saccharomyces cerevisiae at concentrations in the mM range. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM salicylic acid, or any ranges or subranges therebetween. In some embodiments, the feedstock comprises at least 10 μM salicylic acid.

In some embodiments, the feedstock comprises 4-hydroxybenzaldehyde. It may also be referred to as p-Hydroxybenzaldehyde, 4-Formylphenol, or p-Formylphenol. 4-Hydroxybenzaldehyde is a hydroxybenzaldehyde that is benzaldehyde substituted with a hydroxy group at position C-4. It is found in the benzoate degradation via hydroxylation, bisphenol A degradation, toluene and xylene degradation, and biosynthesis of phenylpropanoids pathways. 4-Hydroxybenzaldehyde has a role as a plant metabolite, a mouse metabolite and a dopamine beta-monooxygenase inhibitor. It is also a known microbial inhibitor of, e.g., oleaginous yeast. See Wang et al., “Inhibitor degradation and lipid accumulation potentials of oleaginous yeast Trichosporon cutaneum using lignocellulose feedstock” Bioresource Technology 2016;218:892-901 (herein incorporated by reference) showing inhibition of Trichosporon cutaneuin at 16 mM concentration. See also Hu et al., “Effects of biomass hydrolysis by-products on oleaginous yeast Rhodosporidium toruloides” Bioresource Technology 2009;100(20):4843-4847 (herein incorporated by reference) showing the inhibition of Rhodosporidium toruloides at 9 mM concentration. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM 4-hydroxybenzaldehyde, or any ranges or subranges therebetween. In some embodiments, the feedstock comprises at least 10 μM 4-hydroxybenzaldehyde. In some embodiments, the feedstock comprises about 10 μM-10 mM 4-hydroxybenzaldehyde. In some embodiments, the feedstock comprises about 10-100 μM 4-hydroxybenzaldehyde.

In some embodiments, the feedstock comprises furfural. Furfural is also known as furan-2-carboxaldehyde, feral, furfuraldehyde, 2-furaldehyde, and pyromucic aldehyde. Within the art, furfural is a well-known and potent inhibitor of microbial fermentation. For example, Banergee et al. (“Inhibition of glycolysis by furfural in Saccharomyces cerevisiae,” Eur J Appl Microbiol Biotechnol 1981;11:226-8) found that furfural inhibited ethanol production by S. cerevisiae at as low as 0.5 g/L (˜10 mM) and complete inhibition occurred at 4 g/L (˜40 mM). See also, Sitepu et al., “Carbon source utilization and inhibitor tolerance of 45 oleaginous yeast species,” J Ind Microbiol Biotechnol 2014;41(7):1061-1070. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM furfural, or any ranges or subranges therebetween. In some embodiments, the feedstock comprises at least 10 μM furfural.

In some embodiments, the feedstock comprises 5-(hydroxymethyl)furfural (“HMF”). This compound is also known as 5-hydroxymethyl-2-furaldehyde, 5-Hydroxymethyl-2-furancarboxaldehyde, HMF, and 5-HMF. HMF is well known in the art as a potent inhibitor of microbial fermentation. See Klinke et al., “Inhibition of ethanol-producing yeast and bacteria by degradation products produced during pre-treatment of biomass,” Appl. Microbiol. Biotechnol. 66 (2004) 10-26, which reports significant inhibition of growth and ethanol production at concentrations as low as 8 mM HMF. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM 5-hydroxymethyl-furfural, or any ranges or subranges therebetween. In some embodiments, the feedstock comprises at least 10 μM 5-hydroxymethyl-furfural. In some embodiments, the feedstock comprises about 1 μM-1 mM 5-hydroxymethyl-furfural.

In some embodiments, the feedstock comprises propamocarb. Propamocarb is a known systemic fungicide. See, e.g., ebi.ac.uk/chebi/searchId.do?chebiId=CHEBI:82033. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM propamocarb, or any ranges or subranges therebetween. In some embodiments, the feedstock comprises at least 10 μM propamocarb.

In some embodiments, the feedstock comprises alpha-hydroxyisocaproic acid. alpha-hydroxyisocaproic acid is a known antibacterial and antifungal agent. See, e.g., Nieminen et al., “DL-2-hydroxyisocaproic acid attenuates inflammatory responses in a murine Candida albicans biofilm model,” Clin Vaccine Innnunol 2014:21(9):1240-1245. In some embodiments, the feedstock comprises at least 10 μM, 20 μM, 30 μM, 40 μM, 50 μM, 60 μM, 70 μM, 80 μM, 90 μM, 100 μM, 200 μM, 300 μ1M 400 μM, 500 μM, 600 μM,700 μM, 800 μM, 900 μM, 1 mM, 2 rnM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM alpha-hydroxyisocaproic acid, or any ranges or subranges therebetween. In some embodiments, the feedstock comprises at least 10 μM alpha-hydroxyisocaproic acid.

In some embodiments, the feedstock comprises succinic acid-2,2,3,3-d4. The toxicity of succinic acids is known in the art. See, e.g., Aderiye et al., “Toxicity of citric and succinic acids for the pycnidiospores of Botryodiplodia theobromae,” Folia Microbiol 1998;43: 147-150. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM succinic acid-2,2,3,3-d4, or any ranges or subranges therebetween. In some embodiments, the feedstock comprises at least 10 μM succinic acid-2,2,3,3-d4.

In some embodiments, the feedstock comprises xylitol, a known antifungal agent. See, e.g., Talattof et al., “Antifungal Activity of Xylitol against Candida albicans: An in vitro Study,” J Contemp Dent Pract 2018; 19(2):125-129. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM xylitol, or any ranges or subranges therebetween. In some embodiments, the feedstock comprises at least 10 μM xylitol.

In some embodiments, the feedstock comprises citric acid. Citric acid is a known microbial inhibitor. See, e.g., Hassan et al., “Effect of some organic acids on some fungal growth and their toxins production,” Int J Advances Biol 2015; 2(1):1-11. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM citric acid, or any ranges or subranges therebetween. In some embodiments, the feedstock comprises at least 10 μM citric acid.

In some embodiments, the feedstock may comprises caproic acid. Also known as hexanoic acid, caproic acid is the carboxylic acid derived from hexane with the chemical formula CH₃(CH₂)₄COOH. It is a short-chain saturated fatty acid that can be created by the metabolic activity of yeasts. It is excreted by yeast during extended lagering at warm temperatures and high yeast cell counts. Salts and esters of hexanoic acid are known as hexanoates or caproates. In some embodiments, the feedstock comprises at least 10 μM, 20 μM, 30 μM, 40μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM caproic acid, or any ranges or subranges therebetween. In some embodiments, the feedstock comprises at least 10 μM caproic acid.

In some embodiments, the feedstock comprises caprylic acid. Caprylic acid is a medium-chain fatty acid with potent antibacterial, antifungal, and anti-inflammatory properties. It is commonly used in the treatment of certain yeast infections and skin conditions. In some embodiments, the feedstock comprises caprylic acid. In some embodiments, the feedstock comprises at least 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μ, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM caprylic acid, or any ranges or subranges therebetween. In some embodiments, the feedstock comprises at least 10 μM caprylic acid.

### USE OF FEEDSTOCK FOR OLEAGINOUS MICROBIAL FERMENTATION

The feedstock of the present disclosure may be used in the fermentation of oleaginous microorganisms for the production of lipids.

### Oleaginous Microbes

The present disclosure provides methods for growing oleaginous microorganisms on feedstock obtained from post-fermentation industrial waste streams. In some embodiments, the oleaginous microorganism is a microalgae, yeast, mold, or bacterium.

The use of oleaginous microorganisms for lipid production has many advantages over traditional oil harvesting methods, e.g., palm oil harvesting from palm plants. For example, microbial fermentation (1) does not compete with food production in terms of land utilization; (2) can be carried out in conventional microbial bioreactors; (3) has rapid growth rates; (4) is unaffected or minimally affected by space, light, or climate variations; (5) can utilize waste products as feedstock; (6) is readily scalable; and (7) is amenable to bioengineering for the enrichment of desired fatty acids or oil compositions. In some embodiments, the present methods have one or more of the aforementioned advantages over plant-based oil harvesting methods.

In some embodiments, the oleaginous microorganism is an oleaginous microalgae. In some embodiments, the microalgae is of the genus Botryococcus, Cylindrotheca, Nitzschia, or Schizochytrium. In some embodiments, the oleaginous microorganism is an oleaginous bacterium. In some embodiments, the bacterium is of the genus Arthrobacter, Acinetobacter, Rhodococcus, or Bacillus. In some embodiments, the bacterium is of the species Acinetobacter calcoaceticus, Rhodococcus opacus, or Bacillus alcalophilus. In some embodiments, the oleaginous microorganism is an oleaginous fungus. In some embodiments, the fungus is of the genus Aspergillus, Mortierella, or Humicola. In some embodiments, the fungus is of the species Aspergillus oryzae, Mortierella isabellina, Humicola lanuginosa, or Mortierella vinacea.

Oleaginous yeast in particular are robust, viable over multiple generations, and versatile in nutrient utilization. They also have the potential to accumulate intracellular lipid content up to greater than 70% of their dry biomass. In some embodiments, the oleaginous microorganism is an oleaginous yeast. In some embodiments, the yeast may be in haploid or diploid forms. The yeasts may be capable of undergoing fermentation under anaerobic conditions, aerobic conditions, or both anaerobic and aerobic conditions. A variety of species of oleaginous yeast that produce suitable oils and/or lipids can be used in accordance with the methods of the present invention. In some embodiments, the oleaginous yeast for use in the methods of the present disclosure naturally produce high (20%, 25%, 50% or 75% of dry cell weight or higher) levels of suitable oils and/or lipids. Considerations affecting the selection of yeast for use in the invention include, in addition to production of suitable oils or lipids for production of food products: (1) high lipid content as a percentage of cell weight; (2) ease of growth; (3) ease of propagation; (4) ease of biomass processing; and (5) glycerolipid profile. In some embodiments, the oleaginous yeast comprise cells that are capable of producing at least 20%, 25%, 50% or 75% or more lipid by dry weight. In other embodiments, the oleaginous yeast contains at least 25-35% or more lipid by dry weight.

Suitable species of oleaginous yeast for use in the present disclosure include, but are not limited to Candida apicola, Candida sp., Cryptococcus albidus, Cryptococcus curvatus, Cryptococcus terricolus, Cutaneotrichosporon oleaginosus, Debaromyces hansenii, Endomycopsis vernalis, Geotrichum carabidarum, Geotrichum cucujoidarum, Geotrichum histeridarum, Geotrichum silvicola, Geotrichum vulgare, Hvphopichia burtonii, Lipomyces lipofer, Lypomyces orentalis, Lipomyces starkeyi, Lipomyces tetrasporous, Pichia mexicana, Rodosporidium sphaerocarpum, Rhodosporidium toruloides Rhodotorula aurantiaca, Rhodotorula dairenensis, Rhodotorula diffluens, Rhodotorula glutinus, Rhodotorula glutinis var. glutinis, Rhodotorula gracilis, Rhodotorula graminis Rhodotorula minuta, Rhodotorula rnucilaginosa, Rhodotorula rnucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri, Trichosporon montevideense, Trichosporon pullulans, Trichosporon sp., Wickerhamomyces canadensis, Yarrowia lipolytica, and Zygoascus meyerae.

In some embodiments, the yeast is of the genera Yarrowia, Candida, Rhodotorula, Rhodosporidium, Metschnikowia, Cryptococcus, Trichosporon, or Lipomyces. In some embodiments, the yeast is of the genus Yarrowia. In some embodiments, the yeast is of the species Yarrowia lipolyfica. In some embodiments, the yeast is of the genus Candida. In some embodiments, the yeast is of the species Candida curvata. In some embodiments, the yeast is of the genus Cryptococcus. In some embodiments, the yeast is of the species Cryptococcus albidus. In some embodiments, the yeast is of the genus Lipomyces. In some embodiments, the yeast is of the species Lipomyces starkeyi. In some embodiments, the yeast is of the genus Rhodotorula. In some embodiments, the yeast is of the species Rhodotorula glutinis. In some embodiments, the yeast is of the genus Metschnikowia. In some embodiments, the yeast is of the species Metschnikowia pulcherrima.

In some embodiments, the oleaginous yeast is of the genus Rhodosporidium. In some embodiments, the yeast is of the species Rhodosporidium toruloides. In some embodiments, the oleaginous yeast is of the genus Lipomyces. In some embodiments, the oleaginous yeast is of the species Lipomyces Starkeyi.

In some embodiments, the oleaginous microorganisms to be grown on the feedstock of the present disclosure are a homogeneous population comprising microorganisms of the same species and strain. In some embodiments, the oleaginous microorganisms to be grown on the feedstock of the present disclosure are a heterogeneous population comprising microorganisms from more than one strain. In some embodiments, the oleaginous microorganisms to be grown on the feedstock of the present disclosure are a heterogeneous population comprising two or more distinct populations of microorganisms of different species.

The present disclosure also provides methods for improving one or more aspects of lipid production for oleaginous microorganisms grown on the disclosed feedstocks. These aspects may include lipid yield, lipid titer, dry cell weight titer, lipid content, and lipid composition. In some embodiments, lipid production may be improved by screening microorganisms on the feedstock. In some embodiments, lipid production may be improved by genetic or metabolic engineering to adapt the microorganism for optimal growth on the feedstock. In some embodiments, lipid production may be improved by varying one or more parameters of the growing conditions, such as temperature, shaking speed, growth time, etc. The oleaginous microorganisms of the present disclosure, in some embodiments, are grown from isolates obtained from nature (e.g., wild-types). In some embodiments, wild-type strains are subjected to natural selection to enhance desired traits (e.g., tolerance of certain environmental conditions such as temperature, inhibitor concentration, pH, oxygen concentration, nitrogen concentration, etc.). For example, a wild-type strain (e.g., yeast) may be selected for its ability to grow and/or ferment in a feedstock of the present disclosure, e.g., a feedstock comprising one or more microorganism inhibitors. In other embodiments, wild-type strains are subjected to directed evolution to enhance desired traits (e.g., lipid production, inhibitor tolerance, growth rate, etc.). In some embodiments, the cultures of microorganisms are obtained from culture collections exhibiting desired traits. In some embodiments, strains selected from culture collections are further subjected to directed evolution and/or natural selection in the laboratory. In some embodiments, oleaginous microorganisms are subjected to directed evolution and selection for a specific property (e.g., lipid production and/or inhibitor tolerance). In some embodiments, the oleaginous microorganism is selected for its ability to thrive on a feedstock of the present disclosure.

In some embodiments, directed evolution of the oleaginous microorganisms generally involves three steps. The first step is diversification, wherein the population of organisms is diversified by increasing the rate of random mutation creating a large library of gene variants. Mutagenesis can be accomplished by methods known in the art (e.g., chemical, ultraviolet light, etc.). The second step is selection, wherein the library is tested for the presence of mutants (variants) possessing the desired property using a screening method. Screens enable identification and isolation of high-performing mutants. The third step is amplification, wherein the variants identified in the screen are replicated. These three steps constitute a “round” of directed evolution. In some embodiments, the microorganisms of the present disclosure are subjected to a single round of directed evolution. In other embodiments, the microorganisms of the present disclosure are subjected to multiple rounds of directed evolution. In various embodiments, the microorganisms of the present disclosure are subjected to 2, 3, 4, 5, 6, 7, 8, 9, 10, 20, 30, 40, 50, 60, 70, 80, 90, or 100 or more rounds of directed evolution. In each round, the organisms expressing the highest level of the desired trait of the previous round are diversified in the next round to create a new library. This process may be repeated until the desired trait is expressed at the desired level.

### Fermentation Methods

The present disclosure provides methods of growing oleaginous microorganisms on the disclosed industrial fermentation waste stream feedstocks. Methods of growing and fermenting oleaginous microorganisms are known in the art and may be employed herein. See, for example, U.S. Publication No. 2019/0100780 and U.S. Publication No. 2014/0137463, each incorporated by reference herein.

In some embodiments, the oleaginous microorganisms are cultivated under batch, fed-batch or continuous cultivation conditions. Specifically, the cultivation may be performed in a bioreactor starting with a batch phase followed by a fed-batch phase or a continuous cultivation phase. The terms “fed batch cell culture” and “fed batch culture,” as used herein, refer to a cell culture wherein the cells, preferably oleaginous, and culture medium are supplied to the culturing vessel initially and additional culture nutrients are fed, continuously or in discrete increments, to the culture during culturing, with or without periodic cell and/or product harvest before termination of culture. A “fed batch method,” refers to a method by which a fed batch cell culture is supplied with additional nutrients. For example, a fed batch method may comprise adding supplemental media according to a determined feeding schedule within a given time period.

Classical batch fermentation is a closed system, wherein the composition of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art.

Continuous fermentation is a system where a defined fermentation medium is added continuously to a bioreactor and an equal amount of conditioned medium is removed simultaneously for processing and harvesting of desired biomolecule products of interest. Continuous fermentation generally maintains the cultures at a constant high density where cells are primarily in log phase growth. Continuous fermentation generally maintains the cultures at a stationary or late log/stationary, phase growth. Continuous fermentation systems strive to maintain steady state growth conditions.

Various methods are known for the optimization of several fermentation parameters, such as carbon sources and their ratio with respect to nitrogen source, temperature, pH, nutrient starvation, incubation, time, agitation, etc. to maximize the oil production from oleaginous yeasts such as Rhodotorula sp. (Saxena et al., Journal of the American Oil Chemists' Society 1998; 75(4): 501-505; Yen et al., Journal of Bioscience and Bio engineering 2012; 114(4): 453-456), Yarrowia sp. (Papanikolaou et al., Applied Microbiology and Biotechnology 2002; 58(3): 308-312; Tsigie et al., Bioresource Technology 2011; 102(19): 9216-9222 and 2012; Rakicka et al., Biotechnology for Biofuels 2015; 8(1): 1- 10), Cryptococcus sp. (El-Fadaly et al., Research Journal of Microbiology 2009; 4(8): 301-313; Yu et al., Bioresource Technology 2011; 102(10), 6134-6140), Rhodosporidium sp. (Kraisintu et al., Kasetsart Journal: Natural Science 2010; 44: 436-445; Wiebe et al., BMC Biotechnology 2012; 12(1), 26), Torulaspora sp. (Leesing et al. 2011) and Trichosporon sp. (Huang et al., Bioresource Technology 2012; 110: 711-714; Huang et al., New Biotechnology 2012; 29(3), 372-378; Chen et al., Biotechnology Letters 2012; 34(6): 1025-1028; Gao et al., Bioresource Technology 2014; 152: 552-556). See also Sitepu et al., Bioresource Technology 2013;144:360-9. Each of the foregoing references is herein incorporated by reference in its entirety.

In some embodiments, the pH of the culture can be controlled by any acid or base, or buffer salt, including, but not limited to sodium hydroxide, potassium hydroxide, ammonia, or aqueous ammonia; or acidic compounds such as phosphoric acid or sulfuric acid in a suitable manner. In some embodiments, the pH is generally adjusted to a value of from 6.0 to 8.5, preferably 6.5 to 8.

In some embodiments, a composition of the invention comprises microorganisms and a particular culture medium. In some cases, one or more microorganisms are cultured in one type of medium first and then transferred to a different type of medium. The oleaginous microorganisms may be pre-cultured prior to inoculation of the feedstock. In some embodiments, a medium suitable for pre-culturing is YPS medium. YPS is composed of 10 g/L yeast extract, 20 g/L peptone, and 30 g/L sucrose. In some embodiments, a medium suitable for pre-culturing oleaginous yeast strains is YPD medium. This medium is suitable for axenic cultures, and a 1 L volume of the medium (pH ˜6.8) can be prepared by addition of 10 g bacto-yeast, 20 g bacto-peptone and 40 g glucose into distilled water.

In some embodiments, the microorganisms may be cultured in aerobic, hypoxic, microaerobic, or anaerobic conditions. In some embodiments, the microorganisms are cultured under aerobic conditions. In order to maintain aerobic conditions, oxygen or oxygen-containing gas mixtures such as, for example, air, are introduced into the culture. It is likewise possible to use liquids enriched with hydrogen peroxide. In some embodiments, the microorganisms are cultured under anaerobic conditions. In some embodiments, the microorganisms may be cultured in light or dark conditions. In some embodiments, the microorganisms are cultured in dark conditions. In some embodiments, the microorganisms may be cultured with shaking. In some embodiments, the shaking is performed at 200 rpm.

In some embodiments, the temperature of the culture may be maintained between 15-45° C. In some embodiments, the temperature of the culture may be between 20° C. and 37° C. In some embodiments, the temperature of the culture may be maintained around 30° C. In some embodiments, the temperature may vary over the course of growth.

In some embodiments, the oleaginous microorganisms of the present disclosure are cultured at a particular concentration. For example, the oleaginous microorganisms may be present in a culture at a concentration of greater than 1, 2, 5, 10, 10², 10³, 10⁴, 10⁵, 10⁶, 10⁷, 10⁸, 10⁹, 10¹⁰, 10¹¹, 10¹², 10¹³ cells/ml.

In some embodiments, the microorganisms are cultured for greater than mid-log phase growth. In some embodiments, the microorganisms are cultured for greater than 1, 5, 10, 15, 20, 25, 30, 40, 45, 50, 55, 60, 70, 75, 80, 90, 100, 150, or 200 days. In some embodiments, the microorganisms are continuously cultured.

### Fermentation Compositions

The present disclosure also provides a fermentation composition comprising a feedstock of the present disclosure, an oleaginous microorganism, and a lipid titer. The feedstock is a feedstock as disclosed herein. The oleaginous microorganism may be any of the oleaginous microorganisms described herein. The lipid may be any of the lipids described herein.

The composition may be characterized based on a number of different parameters. In some embodiments, the composition may be characterized based on parameters of the feedstock. In some embodiments, the composition comprises a feedstock as disclosed herein. For example, in some embodiments, the composition comprises a feedstock comprising at least one oleaginous microbial inhibitor.

In some embodiments, the composition is characterized based on dry cell weight (DCW) titer of oleaginous microorganisms. In some embodiments, the composition comprises at least 0.1 g/L, at least 0.2 g/L, at least 0.3 g/L, at least 0.4 g/L, at least 0.5 g/L, at least 0.6 g/L, at least 0.7 g/L, at least 0.8 g/L, at least 0.9 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 5.5 g/L, or at least 6.0 g/L DCW of oleaginous microorganisms. In some embodiments, the composition comprises at least 10 g/L, 20 g/L, 30 g/L, 40 g/L, 50 g/L, 60 g/L, 70 g/L, 80 g/L, 90 g/L, 100 g/L, or 150 g/L DCW of oleaginous microorganisms. In some embodiments, the composition comprises at least 0.5 g/L DCW of oleaginous microorganisms. In some embodiments, the composition comprises at least 1.0 g/L DCW of oleaginous microorganisms. In some embodiments, the composition comprises at least 2.0 g/L DCW of oleaginous microorganisms. In some embodiments, the composition comprises at least 50 g/L DCW of oleaginous microorganisms. In some embodiments, the composition comprises at least 100 g/L DCW of oleaginous microorganisms.

In some embodiments, the composition may be characterized based on its lipid titer. In some embodiments, the composition comprises at least 0.05 g/L, at least 0.1 g/L, at least 0.2 g/L, at least 0.3 g/L, at least 0.4 g/L, at least 0.5 g/L, at least 0.6 g/L, at least 0.7 g/L, at least 0.8 g/L, at least 0.9 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, or at least 5.0 g/L lipid titer. In some embodiments, the composition comprises at least 10 g/L, at least 20 g/L, at least 30 g/L, at least 40 g/L, at least 50 g/L, at least 60 g/L, at least 70 g/L, at least 80 g/L, at least 90 g/L, at least 100 g/L, at least 110 g/L, at least 120 g/L, at least 130 g/L, at least 140 g/L, or at least 150 g/L lipid titer. In some embodiments, the composition comprises at least at least 0.2 g/L lipid titer. In some embodiments, the composition comprises at least at least 0.5 g/L lipid titer. In some embodiments, the composition comprises at least at least 1.0 g/L lipid titer. In some embodiments, the composition comprises at least at least 50 g/L lipid titer. In some embodiments, the composition comprises at least at least 100 g/L lipid titer.

In some embodiments, the composition may be characterized based on the lipid content of the oleaginous microorganisms. In some embodiments, the oleaginous microorganisms comprised by the composition may comprise at least 5%, at least 10%, at least 15%, at least 20%, at least 25%, at least 30%, at least 35%, at least 40%, at least 45%, at least 50%, at least 55%, at least 60%, at least 65%, or at least 70% w/w lipid content. In some embodiments, the oleaginous microorganisms comprise at least 5% w/w lipid content. In some embodiments, the oleaginous microorganisms comprise at least 10% w/w lipid content. In some embodiments, the oleaginous microorganisms comprise at least 30% w/w lipid content.

In some embodiments, the composition may be characterized based on its overall conversion yield. The theoretical maximum amount of oil that may be obtained per gram of carbon source in the feedstock is approximately 0.33 g oil/ g carbon source. In some embodiments, the overall yield is at least 0.05 g oil/g carbon source in the feedstock. In some embodiments, the overall yield is at least 0.05, 0.06, 0.07, 0.08, 0.09, 0.10, 0.11, 0.12, 0.13, 0.14, 0.15, 0.16, 0.17, 0.18, 0.19, or 0.20 g oil/g carbon source in the feedstock.

### Lipids Produced by Oleaginous Microbial Fermentation

The oleaginous microorganisms of the present disclosure may be used to produce lipids (triacylglycerols, diacylglycerols, monoacylglycerols, fatty acids, etc.) by growth and fermentation on the disclosed feedstocks. The lipids produced by the oleaginous microorganisms may be separated or purified from any other component of the spent medium for downstream use in other applications. Lipid produced by the yeast may be used for producing biofuels therefrom or used as a replacement for palm or other oils in food applications or other applications. In some embodiments, the lipids produced by oleaginous microorganisms grown on feedstock according to the present disclosure may be used for a variety of purposes. Microbial lipids produced by oleaginous microorganisms may be generally referred to as single cell oils (SCOs).

The composition of the SCO may vary depending on the strain of microorganism, feedstock composition, and growing conditions. In some embodiments, the lipids produced by the oleaginous microorganisms of the present disclosure comprise about 90% w/w triacylglycerol with a percentage of saturated fatty acids (% SFA) of about 44%. The most common fatty acids produced by oleaginous microbial fermentation on the present feedstocks are oleic acid (C18:1), stearic acid (C18:0), palmitic acid (C16:0), palmitoleic acid (C16:1), and myristic acid (C14:0).

In some embodiments, the SCO comprises at least 5%, at least 10%, at least 15%, at least 20%, at least 25%, at least 30%, at least 35%, at least 40%, at least 45%, at least 50%, at least 55%, or at least 60% w/w palmitic acid. In some embodiments, the SCO comprises at least 5% w/w palmitic acid. In some embodiments, the SCO comprises at least 10% w/w palmitic acid. In some embodiments the SCO comprises 10-20% w/w palmitic acid. In some embodiments the SCO comprises 13-16% w/w palmitic acid.

In some embodiments, the SCO comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9% or at least 10% w/w palmitoleic acid. In some embodiments, the SCO comprises at least 0.1% w/w palmitoleic acid. In some embodiments, the SCO comprises at least 0.5% w/w palmitoleic acid. In some embodiments, the SCO comprises 0.5-10% w/w palmitoleic acid. In some embodiments, the SCO comprises 1-5% w/w palmitoleic acid.

In some embodiments, the SCO comprises at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% w/w stearic acid. In some embodiments, the SCO comprises at least 1% w/w stearic acid. In some embodiments, the SCO comprises at least 5% w/w stearic acid. In some embodiments, the SCO comprises 5-25% w/w stearic acid. In some embodiments, the SCO comprises 9-21% w/w stearic acid.

In some embodiments, the SCO comprises at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54% at least 55%, at least 56%, at least 57%, at least 58%, at least 59%, or at least 60% w/w oleic acid. In some embodiments, the SCO comprises at least 25% w/w oleic acid. In some embodiments, the SCO comprises at least 30% w/w oleic acid. In some embodiments, the SCO comprises 30-65% w/w oleic acid. In some embodiments, the SCO comprises 39-55% w/w oleic acid.

In some embodiments, the SCO comprises myristic acid (C14:0). In some embodiments, the SCO comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% myristic acid.

In some embodiments, the SCO comprises C18:2 (linoleic acid). In some embodiments, the SCO comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% linoleic acid.

In some embodiments, the SCO comprises C18:3 (linolenic acid). In some embodiments, the SCO comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% linolenic acid.

In some embodiments, the SCO comprises C20:0 (arachidic acid). In some embodiments, the SCO comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% arachidic acid.

In some embodiments, the SCO comprises C24:0 (lignoceric acid). In some embodiments, the SCO comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% lignoceric acid.

In some embodiments, the SCO lipid composition is similar to that of plant-derived crude palm oil. In some embodiments, the SCO lipid composition is at least 50%, at least 60%, at least 70%, at least 80%, or at least 90% similar to the lipid composition of palm oil. In some embodiments, the SCO may be used as a palm oil substitute or alternative. In some embodiments, the SCO may be used in the manufacture of any product for which palm oil can be employed. For example, in some embodiments, the SCO may be used in the production of soap bases, detergents, and oleochemicals. In some embodiments, the SCO may be used in the production of food products.

### EXAMPLES

### Example 1: Detection of Inhibitors in Exemplary Post-Fermentation Industrial Feedstock

Exemplary post-fermentation industrial feedstock, syrup from corn stillage, was obtained from a microbial ethanol production waste stream (see FIG. 2A). Such waste streams have not previously been used for oleaginous microbial fermentation, because of concerns regarding low nutrient content and the presence of inhibitors that would prevent microbial growth. To determine the presence of inhibitors, compounds within the feedstock were identified by an untargeted, polar metabolites screen using liquid chromatography with tandem mass spectrometry (LC-MS-MS). The identified metabolites were screened against known inhibitors to select molecules that presented potential inhibitory properties for yeast growth and lipid production.

### LC-MS-MS

The sample was extracted in triplicates of 50 μL each to provide a higher number of identified metabolites and to enable prioritization of reproducible constituents. Samples were precipitated with methanol. The resulting metabolite extracts (n=3) were analyzed with both the global polar and hydrophobic LC-MS-MS assays. Data-dependent high resolution MS2 spectra were searched against the NIST17 MS/MS library to putatively identify specific compounds, and the high scoring (RevDot>900) structures were targeted for relative peak height quantification across the triplicates. Background correction was manually applied using a 3X signal-to-noise cutoff with a floor of 10,000 using blank controls acquired throughout the acquisition. A detection consensus was calculated across the triplicates and putative metabolites were excluded from the final results unless the peak was detected in all three replicates.

### Results

Among those inhibitors detected, the following inhibitors were detected with high confidence in two independent feedstock samples:: 5-aminolevulinic acid, propamocarb, alpha-hydroxyisocaproic acid, furfural, succinic acid-2,2,3,3-d4, xylitol, 5-hydroxymethyl-2-furaldehyde, 4-hydroxybenzaldehyde, p-hydroxyphenyllactic acid, salicylic acid, citric acid, and alpha-hydroxyisocaproic acid. These compounds, and their corresponding chemical formulas, are listed in Table 1 below.

TABLE 1
Inhibitors identified via LC-MS-MS within exemplary feedstock.

| Compound name | Chemical formula |
|---|---|
| 5-aminolevulinic acid | C5H9NO3 |
| propamocarb | C9H20N2O2 |
| alpha-hydroxyisocaproic acid | C6H12O3 |
| furfural | C5H4O2 |
| succinic acid-2,2,3,3-d4 | C4H2D4O4 |
| xylitol | C5H12O5 |
| 5-hydroxymethyl-2-furaldehyde | C6H6O3 |
| 4-hydroxybenzaldehyde | C7H6O2 |
| p-hydroxyphenyllactic acid | C9H10O4 |
| salicylic acid | C7H6O3 |
| citric acid | C6H8O7 |

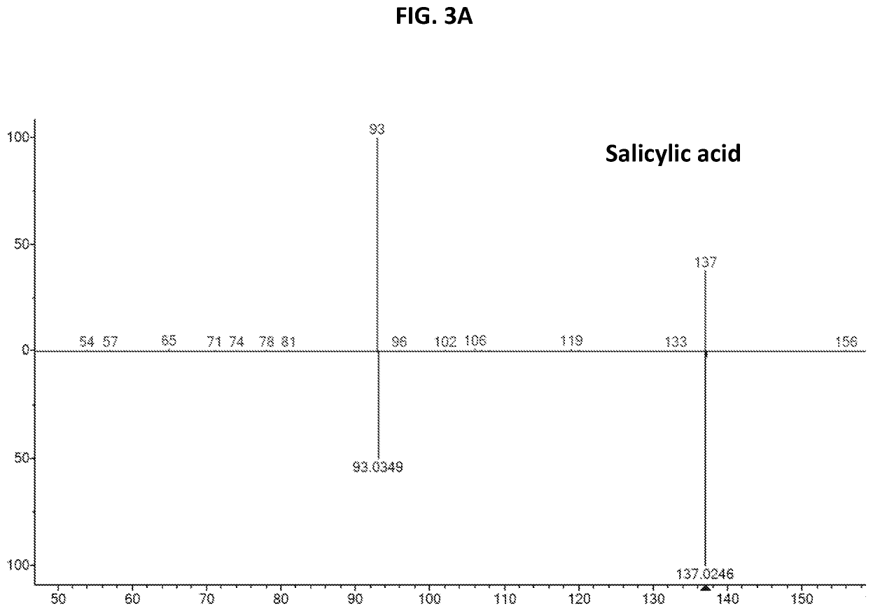

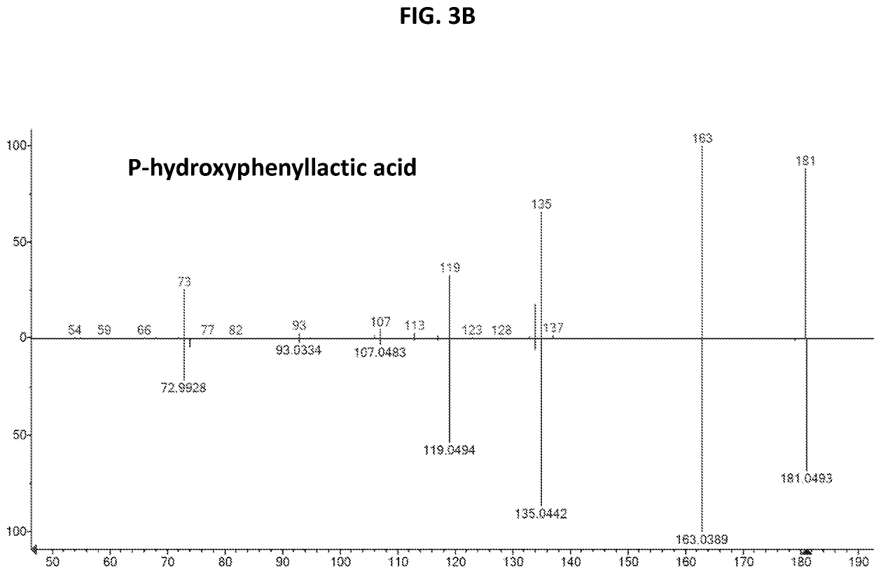

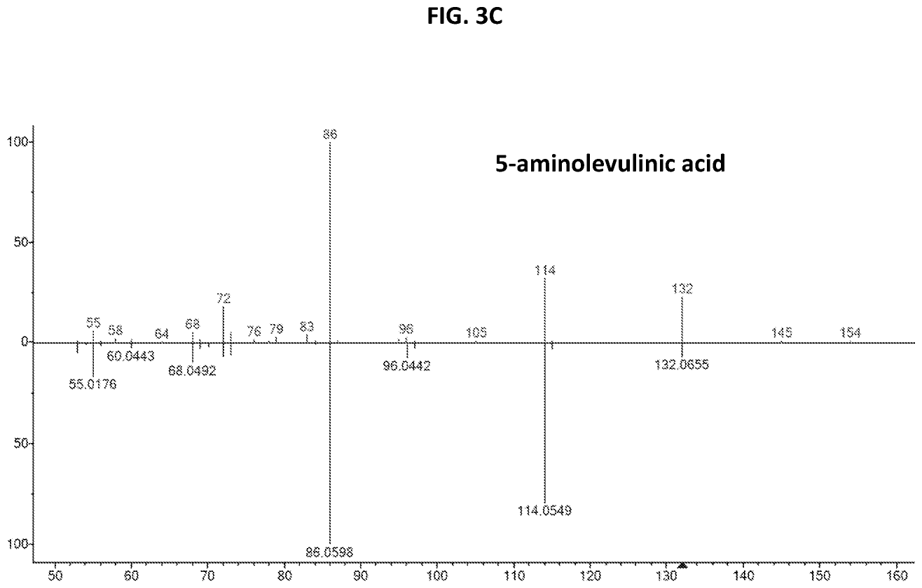

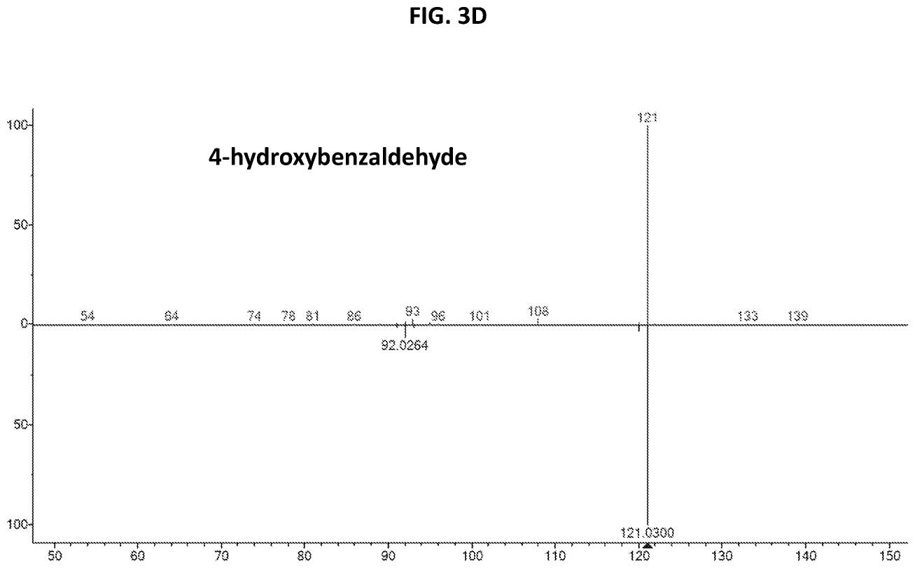

Exemplary MS spectra for four of these inhibitors—salicylic acid, p-hydroxyphenyllactic acid, 5-aminolevulinic acid, and 4-hydroxybenzaldehyde—are shown in FIG. 3A-3D, respectively. In these figures, the bars above the axis represent the reference spectrum for the inhibitor, while the bars below the axis represent the observed spectrum for the inhibitor within the feedstock.

### Example 2: Quantification of Inhibitor Concentration in Exemplary Feedstock

The exemplary feedstock of Example 1 was further tested to determine the concentration of 5-aminolevulinic acid, 5-hydroxymethyl-2-furaldehyde, and 4-hydroxybenzaldehyde via mass spectrometry. Using the same LC-MS-MS method employed in Example 1, a separate panel of standards were run with inhibitors of interest spiked in at known concentrations. These known concentrations of inhibitors were used to generate chromatogram peak standards. The size of the original sample peaks were then translated into concentration ranges based on these standards, allowing for an estimate of the inhibitor concentration range within the original sample.

The results of this analysis are shown in Table 2 below.

TABLE 2
Concentration ranges for select inhibitors within exemplary feedstock.

| Inhibitor | Ion Type | Concentration range |
|---|---|---|
| 5-Aminolevulinic acid | [M + H]+ | 10-200 μM |
| 5-Hydroxymethyl-2- | [M + H]+ | 1 μM-1 mM |
| furaldehyde | | |
| 4-Hydroxybenzaldehyde | [M + H]+ | 10-100 μM |

Based on the results of this analysis, the exemplary feedstock was determined to comprise 5-aminolevulinic acid within a concentration range of 10-200 μM, 5-hydroxymethyl-2-furaldehyde within a range of 1 μM-1 mM, and 4-hydroxybenzaldehyde within a range of 10-100 μM. Each of these compounds is known in the art to exhibit inhibitory effects on microbial growth, as described in the “Inhibitors” section herein. See, e.g., Kamp et al., “Antifungal effect of 5-aminolevulinic acid PDT in Trichophyton rubrum,” Mycoses 2005; 48(2):101-7; Wang et al., “Inhibitor degradation and lipid accumulation potentials of oleaginous yeast Trichosporon cutaneum using lignocellulose feedstock” Bioresource Technology 2016; 218:892-901; Hu et al., “Effects of biomass hydrolysis by-products on oleaginous yeast Rhodosporidium toruloides” Bioresource Technology 2009; 100(20):4843-4847; and Klinke et al., “Inhibition of ethanol-producing yeast and bacteria by degradation products produced during pre-treatment of biomass,” Appl. Microbiol. Biotechnol. 66 (2004) 10-26. As such, these concentrations suggest that some synergistic inhibitory effects would be expected to impede oleaginous microbial growth.

### Example 3: Nutritional Evaluation of Exemplary Feedstock

Exemplary feedstocks of the disclosure were evaluated for nutritional content. The analyses were performed to detect content of metals, fats, carbohydrates, protein, sugar, and salt in four exemplary feedstocks of the disclosure and, as a control, a yeast broth (YPD). The four exemplary feedstocks were sample 1—corn stillage syrup; sample 2—corn thin stillage; sample 3—corn whole stillage; and sample 4—corn stillage pre-blend. Before analysis, samples 1,2, and 3 were diluted to similar working concentrations.

### Results

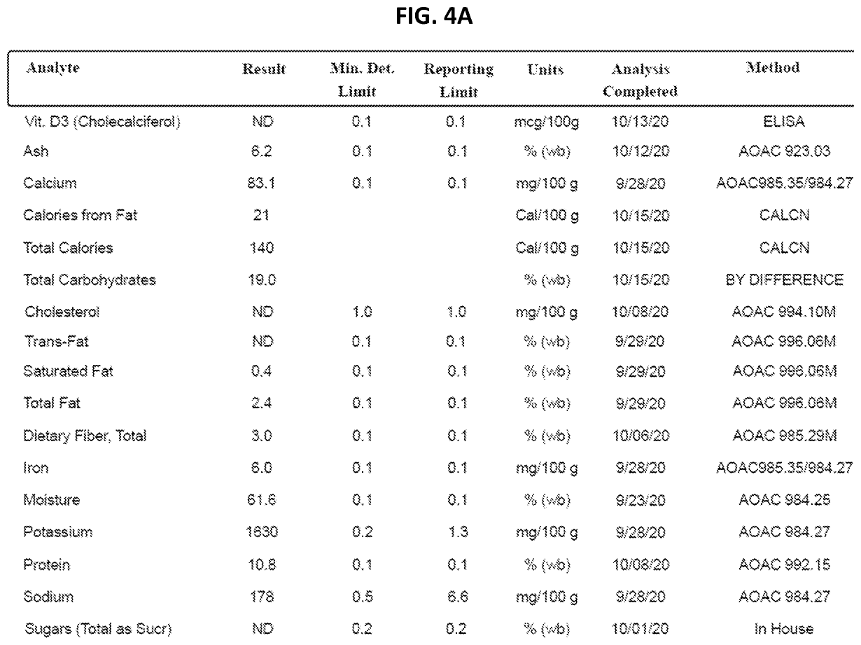

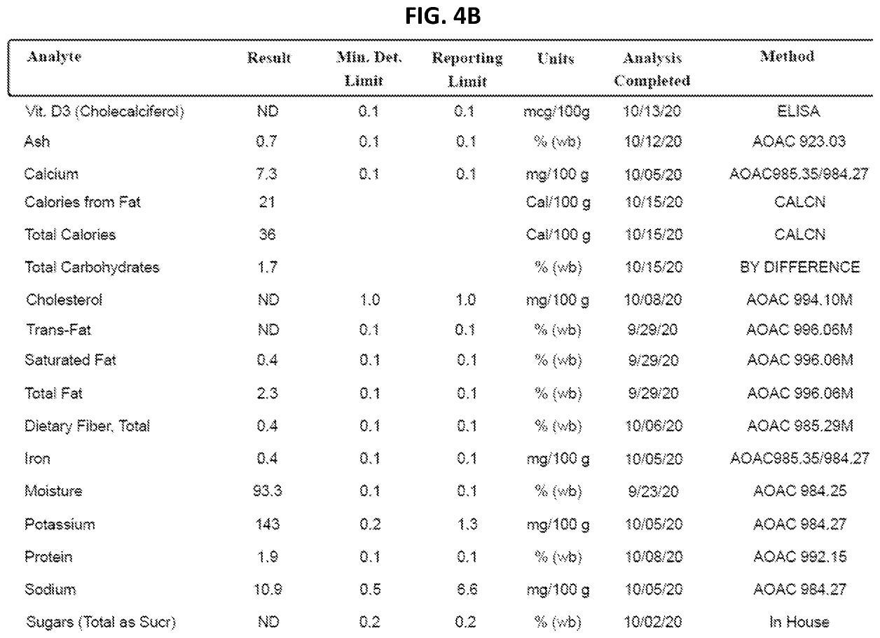

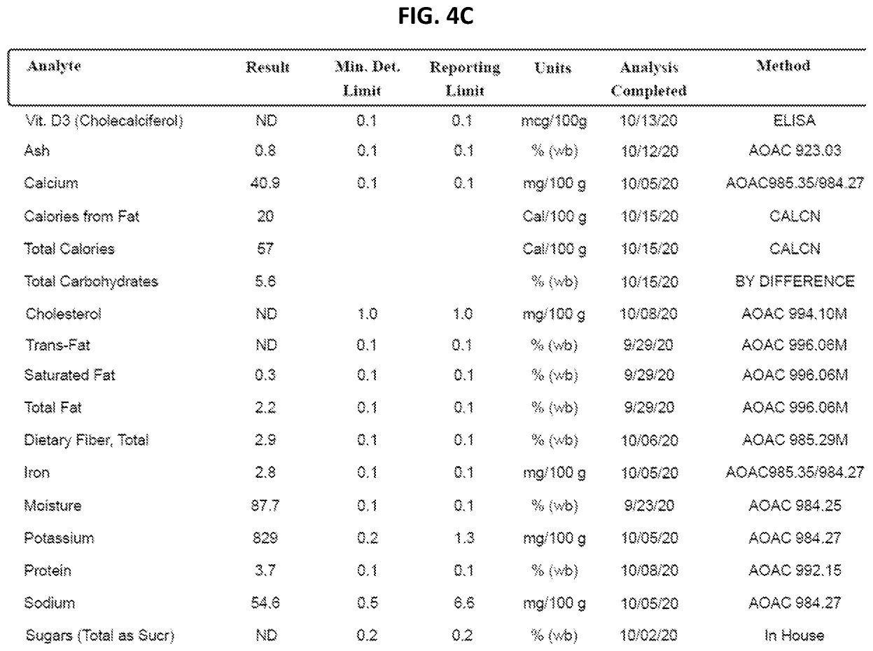

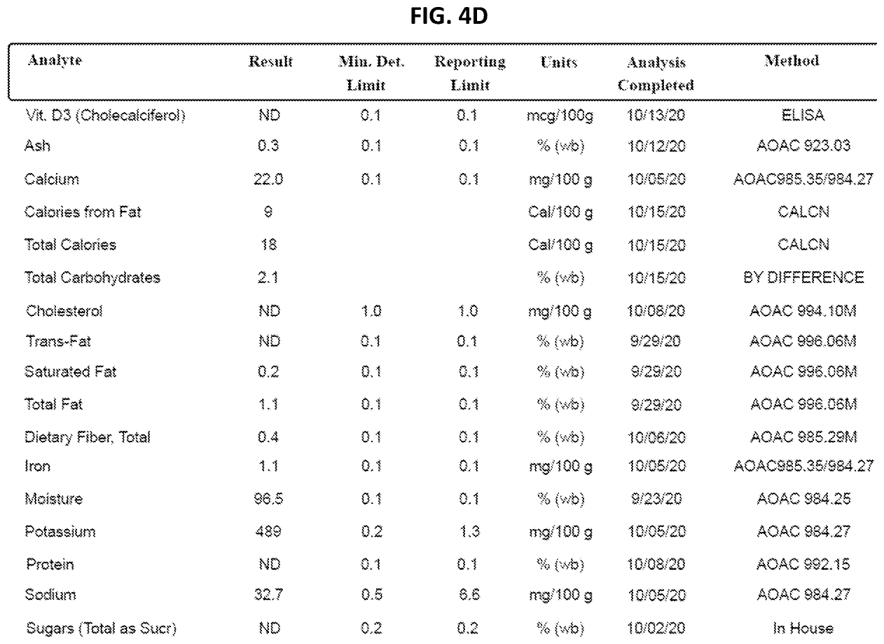

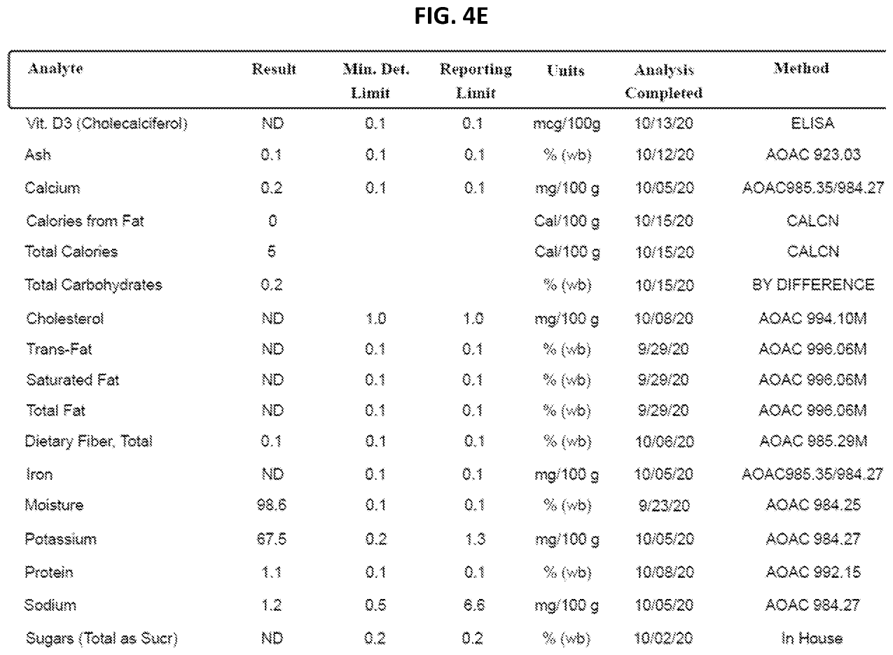

FIGS. 4A-4D show the nutritional analyses for four different exemplary post-fermentation feedstocks of the present disclosure, while FIG. 4E shows the results of the nutritional analysis for the yeast broth control. The results for sample 1 (corn stillage syrup) are shown in FIG. 4A; sample 2 (corn thin stillage), FIG. 4B; sample 3 (corn whole stillage), FIG. 4C; and sample 4 (corn stillage pre-blend), FIG. 4D. A comparison between these exemplary feedstocks of the disclosure and the yeast broth reveals that the feedstocks comprise a much higher content of metals and salts (calcium, potassium, iron, sodium) than in the yeast broth control. The feedstocks also comprise little to no carbohydrates and sugar.

Therefore, prior to the disclosure of the present application, the poor nutritional content, the high concentration of metals and salts, and the presence of inhibitors in these feedstocks (as shown in Examples 1 and 2) would have suggested to the skilled person that these feedstocks would be poor candidates for supporting microbial fermentation.

### Example 4: Rhodosporidium Toruloides Fermentation on Industrial Fermentation Waste Stream Feedstock

The feedstock of Example 1 was used in the fermentation of an exemplary oleaginous yeast, Rhodosporidium toruloides.

### Feedstock Preparation

Corn stillage syrup has a high viscosity and requires centrifugation and/or dilution prior to use as a feedstock. Five preparations of the feedstock were made. Preparation A comprised a 10% v/v dilution of the feedstock with water (10% syrup, 90% water), spun down at 5000 g for 10 min to remove insoluble components. Preparation B comprised the feedstock spun down at 5000 g for 10 min to remove insoluble components without dilution. Preparation C comprised a 10% dilution of the feedstock with water (10% syrup, 90% water). Preparation D comprised a 20% dilution of the feedstock with water (20% syrup, 80% water). Preparation E comprised a 30% dilution of the feedstock with water (30% syrup, 70% water). These preparations were compared to a theoretically optimized medium comprising a C/N ratio of 100 (control preparation).

### Culturing R. Toruloides

100 mL of each preparation was autoclaved in a 250 mL flask and inoculated to OD600=1 with precultures of R. toruloides cultivated overnight in YPS (10 g/L yeast extract, 20 g/L peptone, 30 g/L sucrose). The cultures were shaken at 200 rpm, 30° C. for 6 to 9 days, and the cells were harvested by centrifugation.

### Determination of Biomass and Lipid Content

The harvested cells were dried in a vacuum oven at 60° C. until their mass was stable. The cells were weighed and lipids were subsequently extracted by acid lysis followed by chloroform:methanol separation. In short, 400 mg of powdered biomass were mixed with 3.2 mL of 4 M hydrochloric acid and incubated at 55° C. for 2h. 8 mL of chloroform:methanol 1:1 were added. The solution was shaken vigorously for 3h and centrifuged at 6000 g for 15 min. The chloroform layer was separated and 4 mL of pure chloroform were added. The solution was shaken vigorously for 30 min, spun down again, and the chloroform layer was separated. The samples in chloroform were dried under nitrogen stream until mass was constant and mass was recorded.

Corrections were applied to the measured lipid titers in order to account for lipids already present in the feedstock preparations. Lipids from the “blank” preparations were extracted as described above for the cultures, and the amount of lipids in the blank was subtracted from the amount of lipids in the cultures in order to get the actual amount of lipids produced by R. toruloides. This process provides a low conservative estimate of the lipids produced by the microorganisms, as the yeast consume some portion of the lipids present in the feedstock. For Preparation A and Preparation B, the lipids were separated via centrifugation of the broth prior to inoculation, so no correction was applied.

### Results

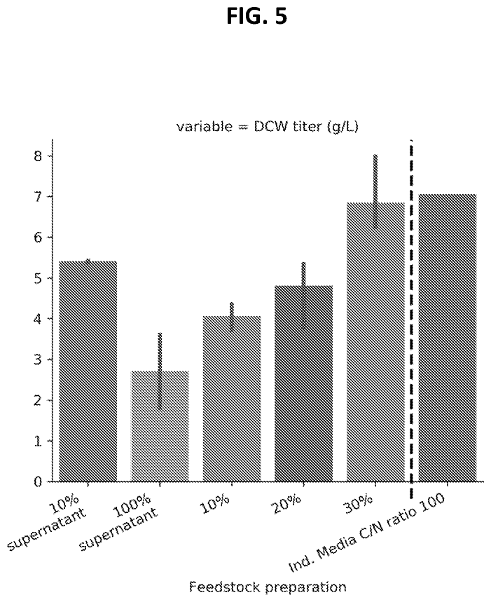

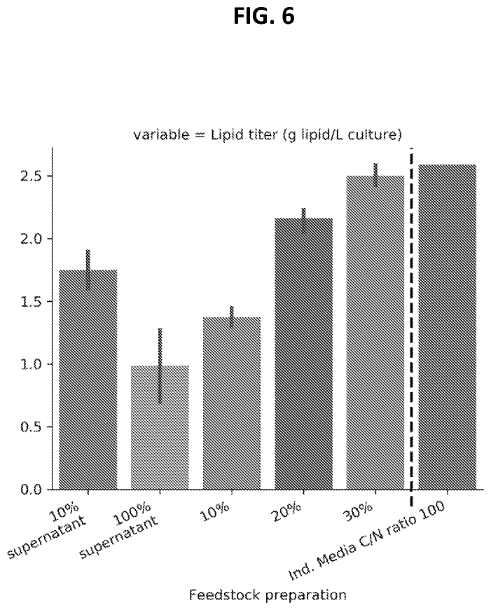

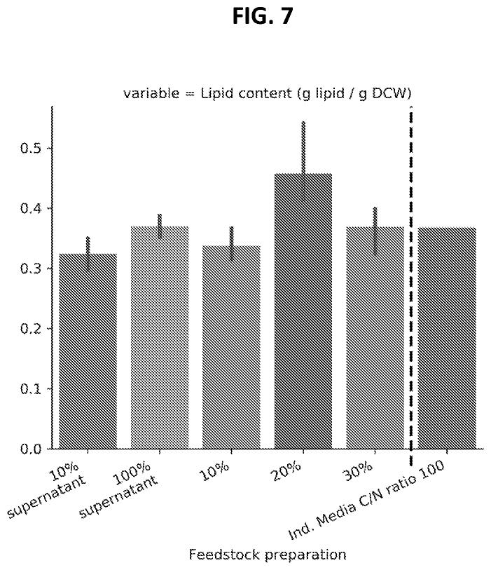

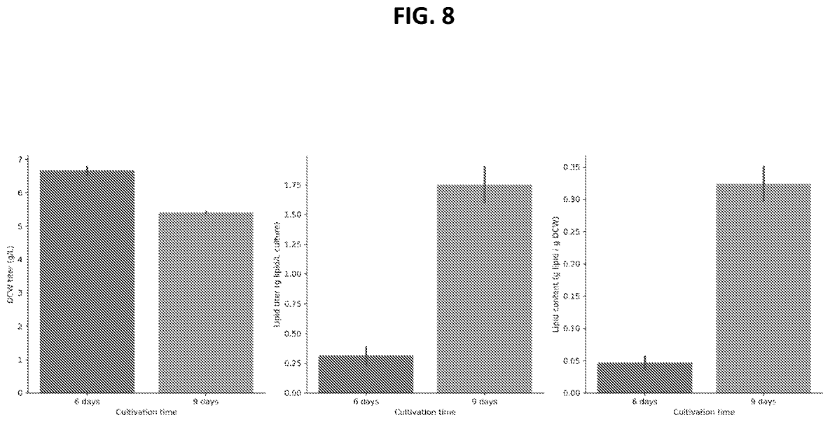

Results of the culture of R. toruloides in the exemplary feedstock of Example 1 are shown in FIGS. 3-6. These results demonstrate that various preparations of the feedstock were able to support oleaginous microbial growth and lipid production. The feedstock contains high concentrations of glycerol, making it viscous and decreasing oxygen transfer. As such, because of the high viscosity of the feedstock, it is impractical for use as is in a shaken vessel, which is why preparation B produced lower biomass production than the other preparations. However, dilutions of the feedstock and clarification by centrifugation (removal of solids) allow for good biomass and lipid production. Increased concentrations of feedstock lead to increasing biomass production and lipid titer (preparations C, D, and E), until viscosity limits oxygen and nutrient transfer (preparation B). Preparation E performed as well as the optimized control preparation, as shown in the results in FIG. 5-7. FIG. 8 demonstrates that lipid content can be optimized by adjusting the time period of fermentation.

### Example 5: Media Formulation, Fermentation, and Lipid Extraction

### Media Formulation

For the following examples, fermentation feedstocks were acquired in 4 separate formulations—i.e., as whole stillage, thin stillage, clarified stillage, and syrup. Fermentation media formulations were optionally diluted in deionized water at various fractions (a “10% feedstock” medium indicates a 1:9 ratio of feedstock fraction to water). Feedstocks were optionally fractionated into supernatant and solid fractions via centrifugation.

### Fermentation

To prepare inocula, yeast strains were propagated at 30° C., 200rpm, for 28 hours in yeast extract-peptone-dextrose (YPD) medium composed of 10 g/L yeast extract, 20 g/L peptone, and 20 g/L dextrose. Cultures were washed of residual nutrients before inoculating 100 mL of the exemplary feedstock to a starting 0D600 of 1.0.

Fermentations were run in 250 mL baffled flasks within an orbital shaker incubator at 200 rpm and 30° C. During harvest, cultures were centrifuged at 4700g for 10 minutes to pellet, resuspended in 20 mL deionized water, and centrifuged again to yield a washed, wet cell pellet.

### Insoluble Matter Correction and Solvent Pre-Wash Procedure Before Lipid Extraction

Some exemplary feedstocks formulated from post-fermentation waste streams contain insoluble matter that needs to be removed or quantified to result in accurate microbial biomass and lipid content calculations. To correct for this content within the feedstock itself, blank cultures were prepared and collected to assess the carryover weight of insoluble matter in the exemplary feedstock. For feedstocks formulated from corn thin stillage, the biomass was able to be separated from the insoluble matter, such that no correction was required. For feedstocks formulated from post-fermentation media clarified supernatants after centrifugation, the feedstock did not comprise insoluble matter, and no correction was necessary.

For all feedstock formulations, the extraction of native lipids was performed on the wet cell pellet/feedstock mixture using a solvent pre-wash of 20 mL chloroform/methanol mixture (2:1 v/v). The pellet and pre-wash mixture was then incubated in a 50° C. water bath for 10 minutes. Next, it was incubated in a 50° C. shaker at 400 rpm for 45 minutes. Samples were centrifuged at 4700g for 10 minutes to induce phase separation. Chloroform and methanol were carefully decanted. The insoluble matter of the diluted corn stillage syrup feedstock could not be separated from the biomass to obtain a pure wet cell pellet, but the biomass could be separated from the thin stillage insoluble matter using 250 g/L sorbitol for a density gradient. Collected and washed cultures were resuspended in 45 mL 250 g/L sorbitol then centrifuged at 4700×g for 10 minutes. The top layer that formed was the desired biomass, whereas the insoluble matter collected at the bottom. The biomass layer was isolated and washed in 45 mL deionized water to obtain the wet cell pellet.

### Lipid Extraction

Biomass was dried to a constant mass in a vacuum oven. Dry cell weight (DCW) was then measured, with correction for insoluble matter as needed. Dried biomass was lysed with 8 mL 4M HCl at 55° C., mild agitation for two hours and extracted with 8 mL chloroform/methanol mixture (2:1 v/v) at room temperature, 350 rpm for three hours. The mixture was centrifuged at 4700g for 10 minutes. The lower layer of chloroform with extracted lipids was isolated and re-extracted using 4 mL chloroform at room temperature, 350 rpm for 30 minutes. Chloroform was evaporated to finalize the lipid extraction. Oil titer was then calculated, with correction for contributions from insoluble matter as needed. Lipid content was determined by dividing oil titer by dry cell weight.

### Example 6: Fermentation of Oleaginous Microorganisms on Exemplary Feedstock

### Feedstock

For the purposes of this example, the exemplary feedstock employed was a 30% corn stillage syrup-based feedstock, comprising 30% v/v corn stillage syrup, with insoluble components removed via centrifugation, diluted in deionized water.

### Microorganisms

Four strains of oleaginous microorganisms were selected to investigate the potential of the exemplary feedstock to support the growth of oleaginous microorganisms: R. toruloides strain A, R. toruloides strain B, Y. lipolytica strain po 1 g, and L. starkeyi strain CBS 1807. As a control, a canonical non-oleaginous yeast, P. pastoris strain X33, was included for comparison.

### Batch Fermentation Results

Fermentations were run in batch format for 6 days. Dry cell weight, oil titer and lipid content were evaluated as described in Example 5.

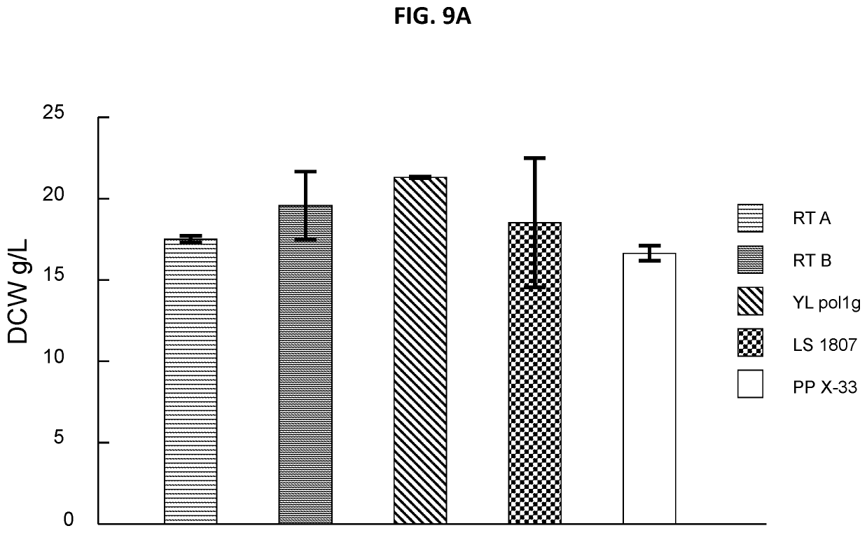

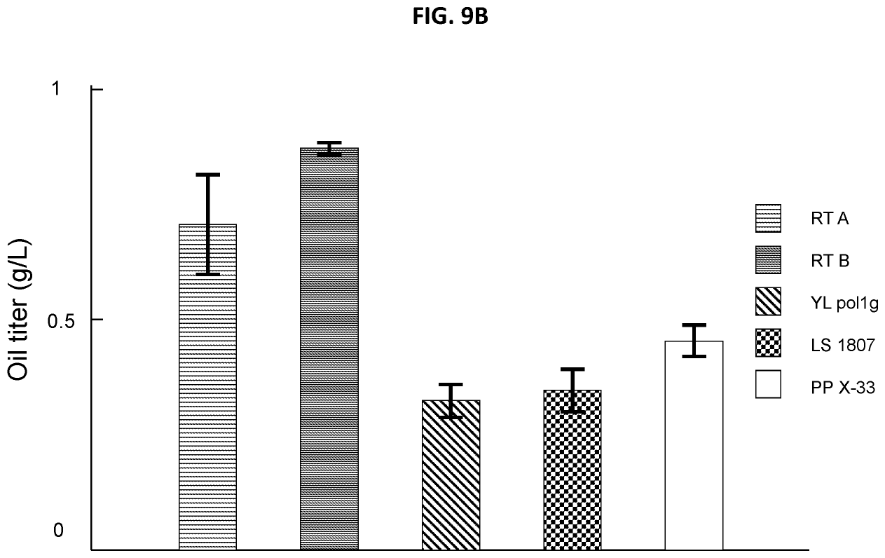

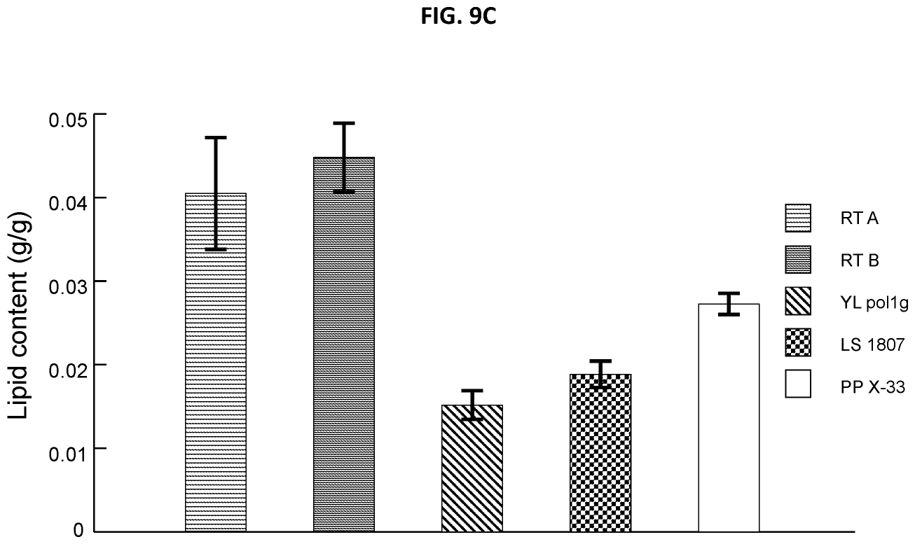

The DCW, oil titer, and lipid content for all five strains is shown in FIGS. 9A-9C. The feedstock supported cell growth for all of the tested yeast strains. The two strains of R. toruloides performed the best in terms of oil titer and lipid content.

Batch Fermentation Results with Glycerol Supplement

50 g/L glycerol was added to the feedstock and fermentations were again run for 6 days. Dry cell weight and oil titer were evaluated and compared to the results of the batch fermentation.

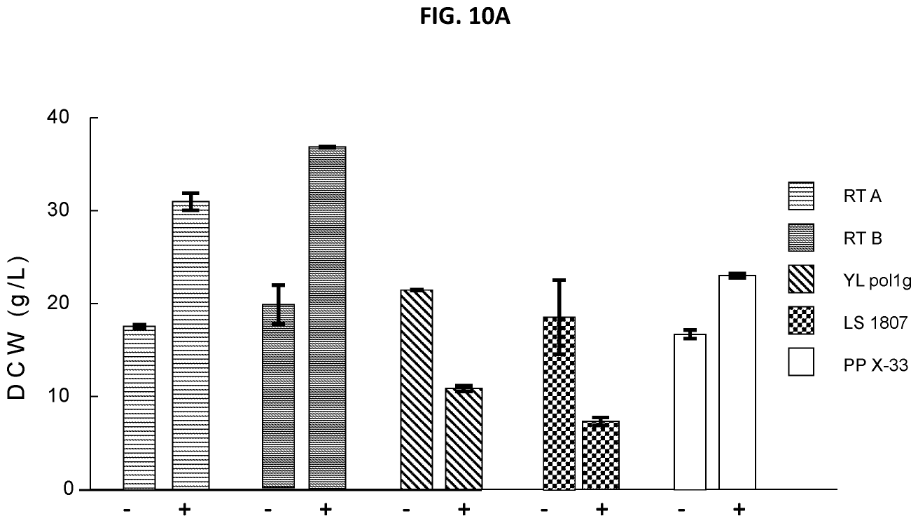

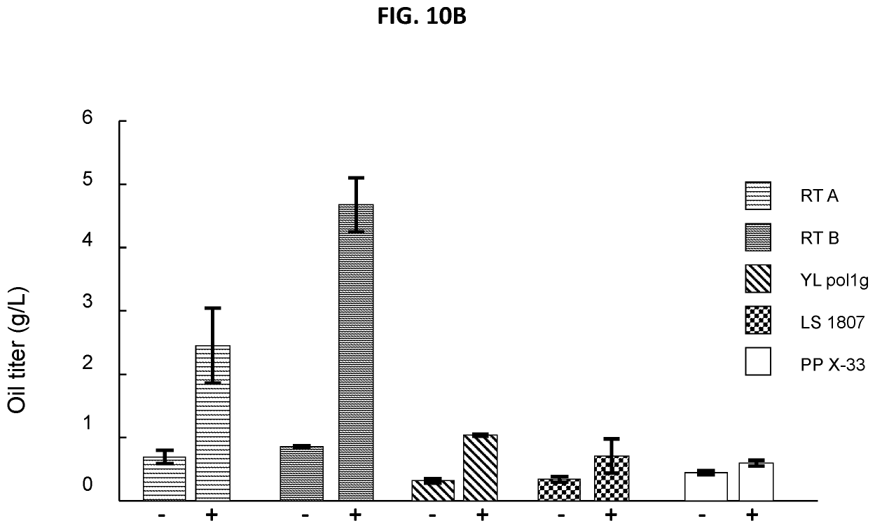

The DCW and oil titer for all five strains are shown in FIGS. 10A-10B. In these figures “−” shows the results for the batch fermentation without added glycerol, while “+” shows the results for the batch fermentation with added glycerol. Surprisingly, both strains of R. toruloides dramatically increased growth and oil production with added glycerol, while Y. lipolytica and L. starkeyi experienced significantly less growth and only marginally improved oil titer.

### Example 7: Fed Batch Fermentation with R. Toruloides

A strain of R. toruloides was tested in a fed-batch fermentation format on two different exemplary feedstocks of the disclosure: 30% stillage and 40% stillage. The 30% and 40% stillage feedstocks were formulated with 30% and 40% corn stillage syrup, respectively, diluted in deionized water. The strain was also grown on defined media as a control. The carbon source for this fed batch fermentation was pure glycerol. The cultures were periodically sampled to measure residual glycerol concentration (via HPLC) and then fed with a bolus of concentrated glycerol (800 g/L) to replenish carbon to 60 g/L.

The fermentations were run for 7 days (˜148 hrs). Total target glycerol (batch+feed) was 281.5 g/L. The cells were then evaluated for DCW, oil titer, lipid content, productivity, and yield. Productivity was calculated by taking the oil titer and dividing by the fermentation time in hours. Yield was calculated by taking the oil titer and dividing by the mass of total glycerol (batch and feed) per liter.

### Results

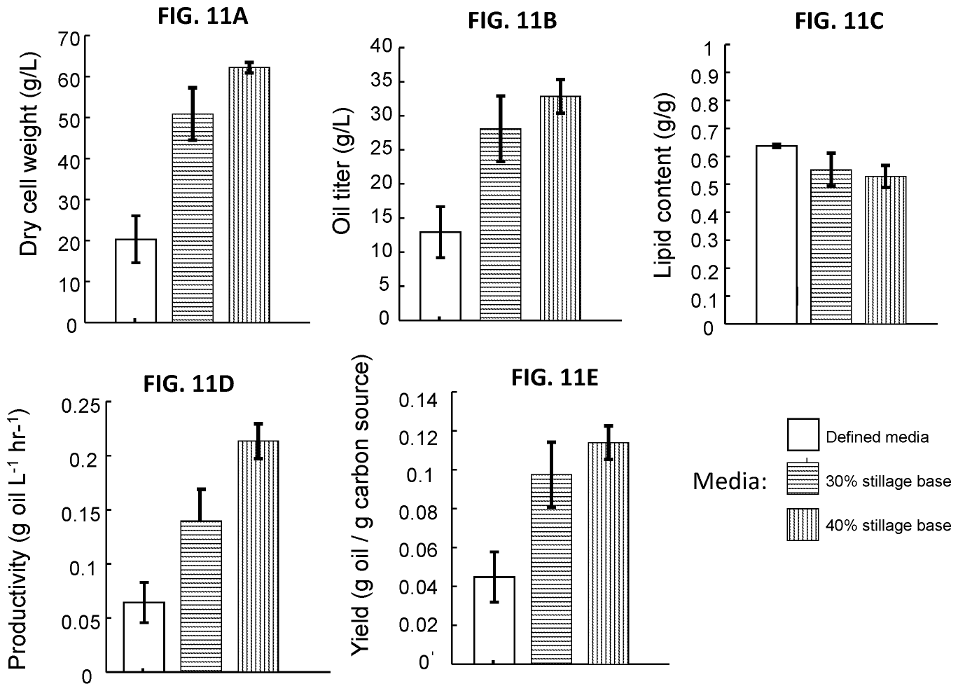

The results of this fed batch experiment in terms of DCW, oil titer, lipid content, are shown in FIGS. 11A-11E. The 40% feedstock outperformed the 30% feedstock which outperformed the defined media control in terms of DCW, oil titer, productivity, and yield. Lipid content, which is calculated by dividing the oil titer by the DCW, was comparable across all three feedstocks.

These results demonstrate that the feedstocks of the present disclosure can be used to produce high oil titers, over 30 g/L for the 40% feedstock condition, in the commercially relevant setting of fed batch fermentation.

### Example 8: Exemplary R. Toruloides Growth on Different Feedstocks and Carbon Sources

### Growth on Different Post-Fermentation Feedstocks

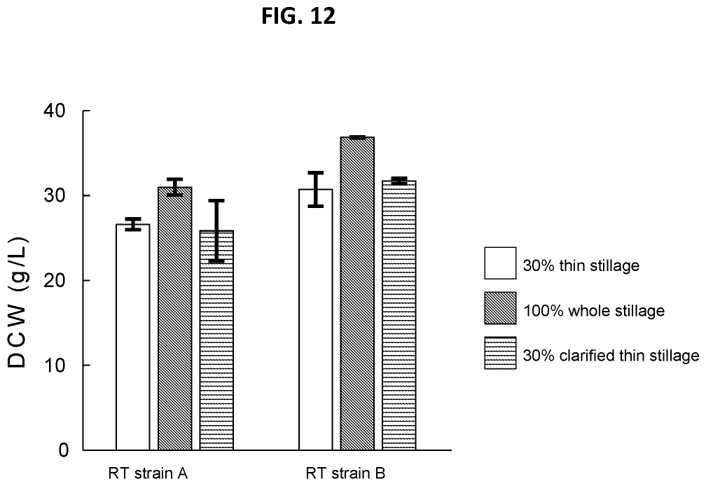

Two exemplary R. toruloides strains, strain A and strain B, were fermented on three exemplary post-fermentation feedstocks of the present disclosure. The feedstocks were: 30% thin stillage—30% thin corn stillage diluted in deionized water; 100% whole stillage; and 30% clarified thin stillage—30% thin corn stillage diluted in deionized water and then clarified via centrifugation. The DCW results shown in FIG. 12 demonstrate that all three of these feedstocks were able to support cell growth for both strains of R. toruloides.

### Growth on Different Carbon Sources

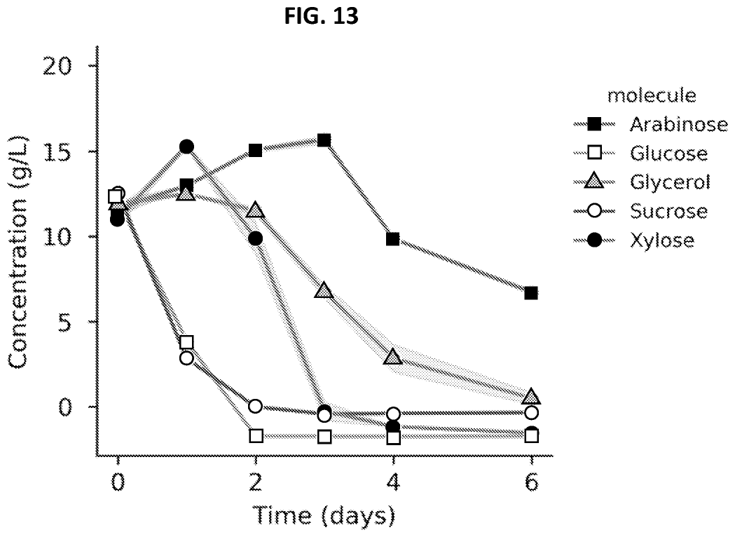

Three exemplary strains of R. toruloides (strains A, B, and C) were grown on yeast peptone (YP) media (20 g/L peptone, 10 g/L yeast extract) with added arabinose, glucose, glycerol, sucrose, and xylose combined to determine the ability and preference of this species to consume different carbon sources. The carbon sources were added to equal initial concentrations of 12 g/L each, with a total carbon content of 60 g/L within the sample. The consumption of these carbon sources was measured via HPLC over time. The results of the analysis demonstrated that all three tested strains of R. toruloides could use any of the five carbon sources as fuel. FIG. 13 is an exemplary graph demonstrating that all five carbon sources were consumed by R. toruloides strain A, with the general trend of preference in terms of consumption being: Glucose>Sucrose>Xylose/Fructose>Glycerol>Arabinose. Note that xylose and fructose co-eluted, such that this curve should be interpreted to comprise both.

These results indicate that three different exemplary strains of R. toruloides were able to utilize a variety of carbon sources as fuel for fermentation.

### Example 9: Evaluation of the Effect of Titrating an Exemplary Inhibitor Found in Post-Fermentation Feedstocks

For the inhibitor 4-hydroxybenzaldehyde, which was found in the exemplary post-fermentation (corn stillage) feedstocks of the present disclosure in Examples 1 and 2, a “spike-in” experiment was performed to evaluate the effect of the inhibitor on the growth and oil production on oleaginous microorganisms. The two strains tested were R. toruloides strain A and Y. lipolytica strain poll g. For this experiment, 4-hydoxybenzaldehyde was deliberated added to YPG media (20 g/L peptone, 10 g/L yeast extract, 60 g/L glycerol) at concentrations of 1 μM, 10 μM, 100 μM, 1 mM, 10 mM and 100 mM in order to assess the direct impact on growth and oil titer for these two strains. A culture without any inhibitor (the “0” condition) was run as a control.

### Results

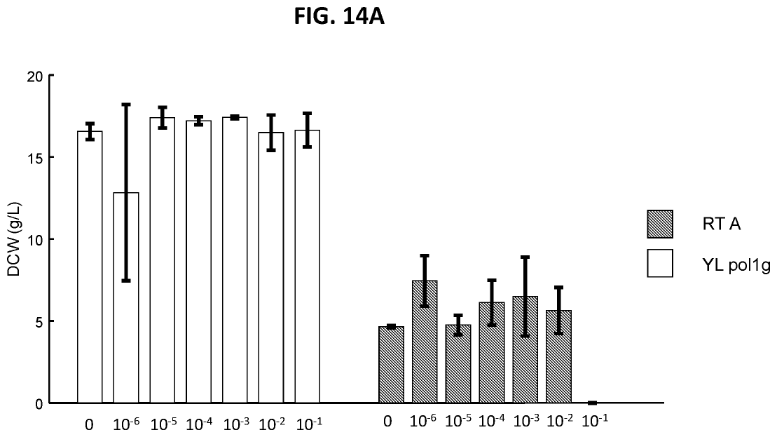

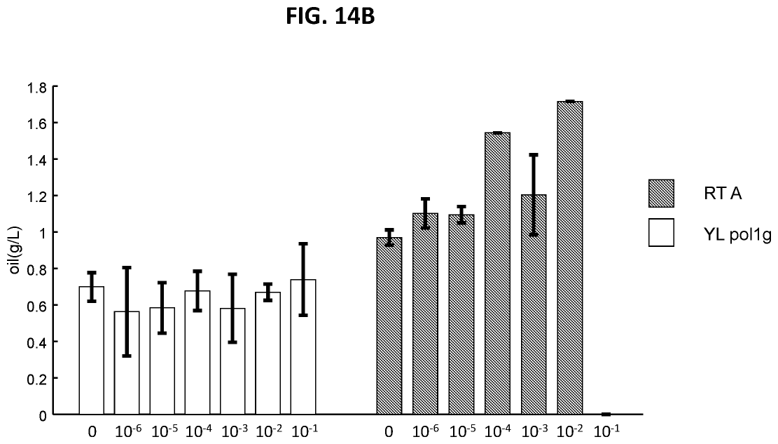

The results for DCW and oil titer are shown in FIGS. 14A-14B, respectively. At the concentrations tested, the inhibitor showed no statistically significant effect on the growth or oil production of the Y. lipolytica strain. The growth of the R. toruloides strain was unaffected at all concentrations, except 100 mM, at which concentration R. toruloides growth was inhibited altogether. Unexpectedly, however, it was observed that increasing concentrations of 4-hydoxybenzaldehyde between 1μM and 10 mM had a lipogenic effect on R. toruloides, generally producing an increase in oil titer at higher concentrations of inhibitor, with a remarkable increase in oil titer of over 50% observed in the 100 μM and 10 mM conditions as compared to the no inhibitor control. These results are particularly relevant in light of the results of Example 2, showing that the concentration of 4-hydroxybenzaldehyde was determined to be in the range of 10-100 μM within the exemplary corn stillage feedstock analyzed therein, which is an inhibitor concentration range within which these lipogenic effects would be expected to occur.

### NUMBERED EMBODIMENTS OF THE INVENTION

Notwithstanding the appended claims, the disclosure sets forth the following numbered embodiments:
1. An oleaginous microbial fermentation broth composition, comprising:
   a) a feedstock comprising at least 10 μM concentration of at least one oleaginous microbial inhibitor;
   b) at least 0.5 grams (g) dry cell weight (DCW) per liter (L) oleaginous microbe titer; and
   c) at least 0.2 g lipid per g DCW lipid content.
2. The composition according to embodiment 1, wherein the oleaginous microbial inhibitor is an acid.
3. The composition according to any one of embodiments 1-2, wherein the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.
4. The composition according to any one of embodiments 1-3, wherein the oleaginous microbial inhibitor is an aldehyde.
5. The composition according to any one of embodiments 1-4, wherein the oleaginous microbial inhibitor is 4-hydroxybenzaldehyde, furfural, or 5-hydroxymethyl-2-furaldehyde.
6. The composition according to any one of embodiments 1-5, wherein the oleaginous microbial inhibitor is an ester.
7. The composition according to any one of embodiments 1-6, wherein the oleaginous microbial inhibitor is propamocarb.
8. The composition according to any one of embodiments 1-7, wherein the oleaginous microbial inhibitor is a sugar alcohol.
9. The composition according to any one of embodiments 1-8, wherein the oleaginous microbial inhibitor is xylitol.
10. The composition according to any one of embodiments 1-9, wherein the composition comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
11. The composition according to any one of embodiments 1-10, wherein the composition comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
12. The composition according to any one of embodiments 1-11, wherein the composition comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
13. The composition according to any one of embodiments 1-12, wherein the composition comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
14. The composition according to any one of embodiments 1-13, wherein the composition comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and
15. The composition according to any one of embodiments 1-14, wherein the composition comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
16. The composition according to any one of embodiments 1-15, wherein the composition comprises at least one of the following oleaginous microbial inhibitors: 5-aminolevulinic acid, 4-hydroxybenzaldehyde, and 5-hydroxymethyl-2-furaldehyde.
17. The composition according to any one of embodiments 1-16, wherein the composition comprises at least two or three of the following oleaginous microbial inhibitors: 5-aminolevulinic acid, 4-hydroxybenzaldehyde, and 5-hydroxymethyl-2-furaldehyde.
18. The composition according to any one of embodiments 1-17, wherein the composition comprises each of the following oleaginous microbial inhibitors: 5-aminolevulinic acid, 4-hydroxybenzaldehyde, and 5-hydroxymethyl-2-furaldehyde.
19. The composition according to any one of embodiments 1-18, wherein the composition comprises 4-hydroxybenzaldehyde.
20. The composition according to any one of embodiments 1-19, wherein the composition comprises at least one of the following: at least 5 mg calcium per 100 g composition; at least 0.4 mg iron per 100 g composition; at least 100 mg potassium per 100 g composition; and at least 10 mg sodium per 100 g composition.
21. The composition according to any one of embodiments 1-20, wherein the composition comprises each of the following: at least 5 mg calcium per 100 g composition; at least 0.4 mg iron per 100 g composition; at least 100 mg potassium per 100 g composition; and at least 10 mg sodium per 100 g composition.
22. The composition according to any one of embodiments 1-21, wherein the oleaginous microbes are oleaginous yeast.
23. The composition according to any one of embodiments 1-22, wherein the oleaginous microbes are oleaginous yeast of the genus Rhodosporidium, Yarrowia, or Lipomyces.
24. The composition according to any one of embodiments 1-23, wherein the oleaginous microbes are oleaginous yeast of the genus Rhodosporidium.
25. The composition according to any one of embodiments 1-24, wherein the oleaginous microbes are oleaginous yeast of the species Rhodosporidium toruloides, Yarrowia lipolytica, or Lipomyces starkeyi.
26. The composition according to any one of embodiments 1-25, wherein the oleaginous microbes are oleaginous yeast of the species Rhodosporidium toruloides.
27. The composition according to any one of embodiments 1-26, wherein the composition comprises at least 5.0 g/L DCW.
28. The composition according to any one of embodiments 1-27, wherein the composition comprises at least 10.0 g/L DCW.
29. The composition according to any one of embodiments 1-28, wherein the composition comprises at least 50.0 g/L DCW.
30. The composition according to any one of embodiments 1-29, wherein the feedstock is a yeast fermentation waste product.
31. The composition according to any one of embodiments 1-30, wherein the feedstock is obtained from a yeast-based bioethanol production waste stream.
32. The composition according to any one of embodiments 1-31, wherein the feedstock is not obtained from food waste or hydrolysate from agricultural waste.
33. The composition according to any one of embodiments 1-32, wherein the feedstock is not obtained from a lignocellulosic biomass hydrolysate.
34. The composition according to any one of embodiments 1-33, wherein the lipid titer is at least 5 g/L.
35. The composition according to any one of embodiments 1-34, wherein the lipid titer is at least 10 g/L.
36. The composition according to any one of embodiments 1-35, wherein the lipid titer is at least 25 g/L.
37. The composition according to any one of embodiments 1-36, wherein the composition comprises a concentration of 4-hydroxybenzaldehyde that induces a higher lipid titer compared to the composition without 4-hydroxybenzaldehyde.
38. The composition according to any one of embodiments 1-37, wherein the lipid content is at least 0.3 g lipid/g DCW.
39. The composition according to any one of embodiments 1-38, wherein the lipid content is at least 0.5 g lipid/g DCW.
40. The composition according to any one of embodiments 1-39, wherein the feedstock is not pre-treated.
41. The composition according to any one of embodiments 1-40, wherein the feedstock is not detoxified, hydrolyzed, or treated with activated charcoal.
42. The composition according to any one of embodiments 1-41, wherein the feedstock is not pre-treated with physical, physico-chemical, chemical, or biological means.
43. The composition according to any one of embodiments 1-42, wherein the composition comprises a carbon source.
44. The composition according to any one of embodiments 1-43, wherein the feedstock is a yeast fermentation waste product, and wherein the composition comprises a carbon source not originally present in the feedstock.
45. The composition according to any one of embodiments 1-44, wherein the composition comprises a C3-C12 carbon source.
46. The composition according to any one of embodiments 1-45, wherein the composition comprises a carbon source selected from arabinose, glucose, glycerol, sucrose, and xylose, and any combination thereof.
47. The composition according to any one of embodiments 1-46, wherein the composition comprises a carbon source, and wherein the carbon source is glycerol.
48. The composition according to any one of embodiments 1-47, wherein the composition comprises at least 10 g/L of a carbon source or a mixture of carbon sources.
49. The composition according to any one of embodiments 1-48, wherein the composition comprises at least 50 g/L of a carbon source or a mixture of carbon sources.
50. The composition according to any one of embodiments 1-49, wherein the oleaginous microbes are R. toruloides, wherein the composition comprises a carbon source, and wherein the concentration of the carbon source in the composition yields a higher lipid titer from the species R. toruloides as compared to a control composition with the species Y. lipolytica or L. starkeyi.
51. An oleaginous microbial fermentation broth composition, comprising:
   a) a feedstock comprising at least 10 μM concentration of at least one oleaginous microbial inhibitor;
   b) at least 10 g/L glycerol;
   c) at least 0.5 grams (g) dry cell weight (DCW) per liter (L) oleaginous microbe titer; and
   d) at least 0.2 g lipid per g DCW lipid content.
52. A method of producing an oleaginous microbial fermentation broth composition, comprising:
   a) growing an oleaginous microbe on a feedstock comprising at least 10 μM concentration of at least one oleaginous microbial inhibitor,
wherein said method results in a microbially produced lipid content of at least 0.2 g lipid/g DCW.
53. The method according to embodiment 52, wherein the oleaginous microbial inhibitor is an acid.
54. The method according to any one of embodiments 52-53, wherein the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.
55. The method according to any one of embodiments 52-54, wherein the oleaginous microbial inhibitor is an aldehyde.
56. The method according to any one of embodiments 52-55, wherein the oleaginous microbial inhibitor is 4-hydroxybenzaldehyde, furfural, or 5-hydroxymethyl furaldehyde.
57. The method according to any one of embodiments 52-56, wherein the oleaginous microbial inhibitor is an ester.
58. The method according to any one of embodiments 52-57, wherein the oleaginous microbial inhibitor is propamocarb.
59. The method according to any one of embodiments 52-58, wherein the oleaginous microbial inhibitor is a sugar alcohol.
60. The method according to any one of embodiments 52-59, wherein the oleaginous microbial inhibitor is xylitol.
61. The method according to any one of embodiments 52-60, wherein the feedstock comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
62. The method according to any one of embodiments 52-61, wherein the feedstock comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
63. The method according to any one of embodiments 52-62, wherein the feedstock comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and
64. The method according to any one of embodiments 52-63, wherein the feedstock comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
65. The method according to any one of embodiments 52-64, wherein the feedstock comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
66. The method according to any one of embodiments 52-65, wherein the feedstock comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
67. The method according to any one of embodiments 52-66, wherein the feedstock comprises at least one of the following oleaginous microbial inhibitors: 5-aminolevulinic acid, 4-hydroxybenzaldehyde, and 5-hydroxymethyl-2-furaldehyde.
68. The method according to any one of embodiments 52-67, wherein the feedstock comprises at least two or three of the following oleaginous microbial inhibitors: 5-aminolevulinic acid, 4-hydroxybenzaldehyde, and 5-hydroxymethyl-2-furaldehyde.
69. The method according to any one of embodiments 52-68, wherein the feedstock comprises each of the following oleaginous microbial inhibitors: 5-aminolevulinic acid, 4-hydroxybenzaldehyde, and 5-hydroxymethyl-2-furaldehyde.
70. The method according to any one of embodiments 52-69, wherein the feedstock comprises 4-hydroxybenzaldehyde.
71. The method according to any one of embodiments 52-70, wherein the feedstock comprises at least one of the following: at least 5 mg calcium per 100 g feedstock; at least 0.4 mg iron per 100 g feedstock; at least 100 mg potassium per 100 g feedstock; and at least 10 mg sodium per 100 g feedstock.
72. The method according to any one of embodiments 52-71, wherein the feedstock comprises each of the following: at least 5 mg calcium per 100 g feedstock; at least 0.4 mg iron per 100 g feedstock; at least 100 mg potassium per 100 g feedstock; and at least 10 mg sodium per 100 g feedstock.
73. The method according to any one of embodiments 52-72, wherein the oleaginous microbes are oleaginous yeast.
74. The method according to any one of embodiments 52-73, wherein the oleaginous microbes are oleaginous yeast of the genus Rhodosporidium, Yarrowia, or Lipomyces.
75. The method according to any one of embodiments 52-74, wherein the oleaginous microbes are oleaginous yeast of the genus Rhodosporidium.
76. The method according to any one of embodiments 52-75, wherein the oleaginous microbes are oleaginous yeast of the species Rhodosporidium toruloides, Yarrowia lipolytica, or Lipomyces starkeyi.
77. The method according to any one of embodiments 52-76, wherein the oleaginous microbes are oleaginous yeast of the species Rhodosporidium toruloides.
78. The method according to any one of embodiments 52-77, wherein the method results in a DCW of at least 5.0 g/L.
79. The method according to any one of embodiments 52-78, wherein the method results in a DCW of at least 10.0 g/L.
80. The method according to any one of embodiments 52-79, wherein the method results in a DCW of at least 50.0 g/L.
81. The method according to any one of embodiments 52-80, wherein the feedstock is a yeast fermentation waste product.
82. The method according to any one of embodiments 52-81, wherein the feedstock is obtained from a yeast-based bioethanol production waste stream.
83. The method according to any one of embodiments 52-82, wherein the feedstock is not obtained from food waste or hydrolysate from agricultural waste.
84. The method according to any one of embodiments 52-83, wherein the feedstock is not obtained from a lignocellulosic biomass hydrolysate.
85. The method according to any one of embodiments 52-84, wherein the method results in a lipid titer of at least 5 g/L.
86. The method according to any one of embodiments 52-85, wherein the method results in a lipid titer of at least 10 g/L.
87. The method according to any one of embodiments 52-86, wherein the method results in a lipid titer of at least 25 g/L.
88. The method according to any one of embodiments 52-87, wherein the feedstock comprises a concentration of 4-hydroxybenzaldehyde that induces a higher lipid titer compared to the feedstock without 4-hydroxybenzaldehyde.
89. The method according to any one of embodiments 52-88, wherein the lipid content is at least 0.3 g lipid/g DCW.
90. The method according to any one of embodiments 52-89, wherein the lipid content is at least 0.5 g lipid/g DCW.
91. The method according to any one of embodiments 52-90, wherein the feedstock is not pre-treated.
92. The method according to any one of embodiments 52-91, wherein the feedstock is not detoxified, hydrolyzed, or treated with activated charcoal.
93. The method according to any one of embodiments 52-92, wherein the feedstock is not pre-treated with physical, physico-chemical, chemical, or biological means.
94. The method according to any one of embodiments 52-93, wherein the feedstock comprises a carbon source.
95. The method according to any one of embodiments 52-94, wherein the feedstock is a yeast fermentation waste product, and wherein the composition comprises a carbon source not originally present in the feedstock.
96. The method according to any one of embodiments 52-95, wherein the composition comprises a C3-C12 carbon source.
97. The method according to any one of embodiments 52-96, wherein the composition comprises a carbon source selected from arabinose, glucose, glycerol, sucrose, and xylose, and any combination thereof.
98. The method according to any one of embodiments 52-97, wherein the composition comprises a carbon source, and wherein the carbon source is glycerol.
99. The method according to any one of embodiments 52-98, wherein the composition comprises at least 10 g/L of a carbon source or a mixture of carbon sources.
100. The method according to any one of embodiments 52-99, wherein the composition comprises at least 50 g/L of a carbon source or a mixture of carbon sources.
101. The method according to any one of embodiments 52-100, wherein the oleaginous microbes are R. toruloides, wherein the composition comprises a carbon source, and wherein the concentration of the carbon source in the composition yields a higher lipid titer from the species R. toruloides as compared to a control composition with the species Y. lipolytica or L. starkeyi.
102. A method of producing microbial lipids from oleaginous microbes, comprising:
   a) providing a feedstock comprising at least 10 μM concentration of at least one oleaginous microbial inhibitor; and
   b) growing the oleaginous microbes on said feedstock, thereby producing microbial lipids.
103. The method according to embodiment 102, wherein the oleaginous microbial inhibitor is an acid.
104. The method according to any one of embodiments 102-103, wherein the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.
105. The method according to any one of embodiments 102-104, wherein the oleaginous microbial inhibitor is an aldehyde.
106. The method according to any one of embodiments 102-105, wherein the oleaginous microbial inhibitor is 4-hydroxybenzaldehyde, furfural, or 5-hydroxymethyl-2-furaldehyde.
107. The method according to any one of embodiments 102-106, wherein the oleaginous microbial inhibitor is an ester.
108. The method according to any one of embodiments 102-107, wherein the oleaginous microbial inhibitor is propamocarb.
109. The method according to any one of embodiments 102-108, wherein the oleaginous microbial inhibitor is a sugar alcohol.
110. The method according to any one of embodiments 102-109, wherein the oleaginous microbial inhibitor is xylitol.
111. The method according to any one of embodiments 102-110, wherein the feedstock comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
112. The method according to any one of embodiments 102-111, wherein the feedstock comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
113. The method according to any one of embodiments 102-112, wherein the feedstock comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and
114. The method according to any one of embodiments 102-113, wherein the feedstock comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
115. The method according to any one of embodiments 102-114, wherein the feedstock comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
116. The method according to any one of embodiments 102-115, wherein the feedstock comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.
117. The method according to any one of embodiments 102-116, wherein the feedstock comprises at least one of the following oleaginous microbial inhibitors: 5-aminolevulinic acid, 4-hydroxybenzaldehyde, and 5-hydroxymethyl-2-furaldehyde.
118. The method according to any one of embodiments 102-117, wherein the feedstock comprises at least two or three of the following oleaginous microbial inhibitors: 5-aminolevulinic acid, 4-hydroxybenzaldehyde, and 5-hydroxymethyl-2-furaldehyde.
119. The method according to any one of embodiments 102-118, wherein the feedstock comprises each of the following oleaginous microbial inhibitors: 5-aminolevulinic acid, 4-hydroxybenzaldehyde, and 5-hydroxymethyl-2-furaldehyde.
120. The method according to any one of embodiments 102-119, wherein the feedstock comprises 4-hydroxybenzaldehyde.
121. The method according to any one of embodiments 102-120, wherein the feedstock comprises at least one of the following: at least 5 mg calcium per 100 g feedstock; at least 0.4 mg iron per 100 g feedstock; at least 100 mg potassium per 100 g feedstock; and at least 10 mg sodium per 100 g feedstock.
122. The method according to any one of embodiments 102-121, wherein the feedstock comprises each of the following: at least 5 mg calcium per 100 g feedstock; at least 0.4 mg iron per 100 g feedstock; at least 100 mg potassium per 100 g feedstock; and at least 10 mg sodium per 100 g feedstock.
123. The method according to any one of embodiments 102-122, wherein the oleaginous microbes are oleaginous yeast.
124. The method according to any one of embodiments 102-123, wherein the oleaginous microbes are oleaginous yeast of the genus Rhodosporidium, Yarrowia, or Lipomyces.
125. The method according to any one of embodiments 102-124, wherein the oleaginous microbes are oleaginous yeast of the genus Rhodosporidium.
126. The method according to any one of embodiments 102-125, wherein the oleaginous microbes are oleaginous yeast of the species Rhodosporidium toruloides, Yarrowia lipolytica, or Lipomyces starkeyi.
127. The method according to any one of embodiments 102-126, wherein the oleaginous microbes are oleaginous yeast of the species Rhodosporidium toruloides.
128. The method according to any one of embodiments 102-127, wherein the method results in a DCW of at least 5.0 g/L.
129. The method according to any one of embodiments 102-128, wherein the method results in a DCW of at least 10.0 g/L.
130. The method according to any one of embodiments 102-129, wherein the method results in a DCW of at least 50.0 g/L.
131. The method according to any one of embodiments 102-130, wherein the feedstock is a yeast fermentation waste product.
132. The method according to any one of embodiments 102-131, wherein the feedstock is obtained from a yeast-based bioethanol production waste stream.
133. The method according to any one of embodiments 102-132, wherein the feedstock is not obtained from food waste or hydrolysate from agricultural waste.
134. The method according to any one of embodiments 102-133, wherein the feedstock is not obtained from a lignocellulosic biomass hydrolysate.
135. The method according to any one of embodiments 102-134, wherein the method results in a lipid titer of at least 5 g/L.
136. The method according to any one of embodiments 102-135, wherein the method results in a lipid titer of at least 10 g/L.
137. The method according to any one of embodiments 102-136, wherein the method results in a lipid titer of at least 25 g/L.
138. The method according to any one of embodiments 102-137, wherein the feedstock comprises a concentration of 4-hydroxybenzaldehyde that induces a higher lipid titer compared to the feedstock without 4-hydroxybenzaldehyde.
139. The method according to any one of embodiments 102-138, wherein the method results in a lipid content of at least 0.3 g lipid/g DCW.
140. The method according to any one of embodiments 102-139, wherein the method results in a lipid content of at least 0.5 g lipid/g DCW.
141. The method according to any one of embodiments 102-140, wherein the feedstock is not pre-treated.
142. The method according to any one of embodiments 102-141, wherein the feedstock is not detoxified, hydrolyzed, or treated with activated charcoal.
143. The method according to any one of embodiments 102-142, wherein the feedstock is not pre-treated with physical, physico-chemical, chemical, or biological means.
144. The method according to any one of embodiments 102-143, wherein the feedstock comprises a carbon source.
145. The method according to any one of embodiments 102-144, wherein the feedstock is a yeast fermentation waste product, and wherein the composition comprises a carbon source not originally present in the feedstock.
146. The method according to any one of embodiments 102-145, wherein the feedstock comprises a C3-C12 carbon source.
147. The method according to any one of embodiments 102-146, wherein the feedstock comprises a carbon source selected from arabinose, glucose, glycerol, sucrose, and xylose, and any combination thereof.
148. The method according to any one of embodiments 102-147, wherein the feedstock comprises a carbon source, and wherein the carbon source is glycerol.
149. The method according to any one of embodiments 102-148, wherein the feedstock comprises at least 10 g/L of a carbon source or a mixture of carbon sources.
150. The method according to any one of embodiments 102-149, wherein the feedstock comprises at least 50 g/L of a carbon source or a mixture of carbon sources.
151. The method according to any one of embodiments 102-150, wherein the oleaginous microbes are R. toruloides, wherein the feedstock comprises a carbon source, and wherein the concentration of the carbon source in the feedstock yields a higher lipid titer from the species R. toruloides as compared to a control feedstock with the species Y. lipolytica or L. starkeyi.

What is claimed is:

1. An oleaginous microbial fermentation broth composition, comprising:
a) a feedstock comprising at least 10 μM concentration of at least one oleaginous microbial inhibitor;
b) at least 0.5 grams (g) dry cell weight (DCW) per liter (L) oleaginous microbe titer; and
c) at least 0.2 g lipid per g DCW lipid content.

2. The composition according to claim 1, wherein the oleaginous microbial inhibitor is an acid.

3. The composition according to claim 1, wherein the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.

4. The composition according to claim 1, wherein the oleaginous microbial inhibitor is an aldehyde.

5. The composition according to claim 1, wherein the oleaginous microbial inhibitor is 4-hydroxybenzaldehyde, furfural, or 5-hydroxymethyl-2-furaldehyde.

6. The composition according to claim 1, wherein the oleaginous microbial inhibitor is an ester.

7. The composition according to claim 1, wherein the oleaginous microbial inhibitor is propamocarb.

8. The composition according to claim 1, wherein the oleaginous microbial inhibitor is a sugar alcohol.

9. The composition according to claim 1, wherein the oleaginous microbial inhibitor is xylitol.

10. The composition according to claim 1, wherein the composition comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

11. The composition according to claim 1, wherein the composition comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

12. The composition according to claim 1, wherein the composition comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

13. The composition according to claim 1, wherein the composition comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

14. The composition according to claim 1, wherein the composition comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

15. The composition according to claim 1, wherein the composition comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl furaldehyde, propamocarb, and xylitol.

16. The composition according to claim 1, wherein the composition comprises at least one of the following oleaginous microbial inhibitors: 5-aminolevulinic acid, 4-hydroxybenzaldehyde, and 5-hydroxymethyl-2-furaldehyde.

17. The composition according to claim 1, wherein the composition comprises at least two or three of the following oleaginous microbial inhibitors: 5-aminolevulinic acid, 4-hydroxybenzaldehyde, and 5-hydroxymethyl-2-furaldehyde.

18. The composition according to claim 1, wherein the composition comprises each of the following oleaginous microbial inhibitors: 5-aminolevulinic acid, 4-hydroxybenzaldehyde, and 5-hydroxymethyl-2-furaldehyde.

19. The composition according to claim 1, wherein the composition comprises 4-hydroxybenzaldehyde.

20. The composition according to claim 1, wherein the composition comprises at least one of the following: at least 5 mg calcium per 100 g composition; at least 0.4 mg iron per 100 g composition; at least 100 mg potassium per 100 g composition; and at least 10 mg sodium per 100 g composition.

21. The composition according to claim 1, wherein the composition comprises each of the following: at least 5 mg calcium per 100 g composition; at least 0.4 mg iron per 100 g composition; at least 100 mg potassium per 100 g composition; and at least 10 mg sodium per 100 g composition.

22. The composition according to claim 1, wherein the oleaginous microbes are oleaginous yeast.

23. The composition according to claim 1, wherein the oleaginous microbes are oleaginous yeast of the genus Rhodosporidium, Yarrowia, or Lipomyces.

24. The composition according to claim 1, wherein the oleaginous microbes are oleaginous yeast of the genus Rhodosporidium.

25. The composition according to claim 1, wherein the oleaginous microbes are oleaginous yeast of the species Rhodosporidium toruloides, Yarrowia lipolytica, or Lipomyces starkeyi.

26. The composition according to claim 1, wherein the oleaginous microbes are oleaginous yeast of the species Rhodosporidium toruloides.

27. The composition according to claim 1, wherein the composition comprises at least 5.0 g/L DCW.

28. The composition according to claim 1, wherein the composition comprises at least 10.0 g/L DCW.

29. The composition according to claim 1, wherein the composition comprises at least 50.0 g/L DCW.

30. The composition according to claim 1, wherein the feedstock is a yeast fermentation waste product.

31. The composition according to claim 1, wherein the feedstock is obtained from a yeast-based bioethanol production waste stream.

32. The composition according to claim 1, wherein the feedstock is not obtained from food waste or hydrolysate from agricultural waste.

33. The composition according to claim 1, wherein the feedstock is not obtained from a lignocellulosic biomass hydrolysate.

34. The composition according to claim 1, wherein the lipid titer is at least 5 g/L.

35. The composition according to claim 1, wherein the lipid titer is at least 10 g/L.

36. The composition according to claim 1, wherein the lipid titer is at least 25 g/L.

37. The composition according to claim 1, wherein the composition comprises a concentration of 4-hydroxybenzaldehyde that induces a higher lipid titer compared to the composition without 4-hydroxybenzaldehyde.

38. The composition according to claim 1, wherein the lipid content is at least 0.3 g lipid/g DCW.

39. The composition according to claim 1, wherein the lipid content is at least 0.5 g lipid/g DCW.

40. The composition according to claim 1, wherein the feedstock is not pre-treated.

41. The composition according to claim 1, wherein the feedstock is not detoxified, hydrolyzed, or treated with activated charcoal.

42. The composition according to claim 1, wherein the feedstock is not pre-treated with physical, physico-chemical, chemical, or biological means.

43. The composition according to claim 1, wherein the composition comprises a carbon source.

44. The composition according to claim 1, wherein the feedstock is a yeast fermentation waste product, and wherein the composition comprises a carbon source not originally present in the feedstock.

45. The composition according to claim 1, wherein the composition comprises a C3-C12 carbon source.

46. The composition according to claim 1, wherein the composition comprises a carbon source selected from arabinose, glucose, glycerol, sucrose, and xylose, and any combination thereof.

47. The composition according to claim 1, wherein the composition comprises a carbon source, and wherein the carbon source is glycerol.

48. The composition according to claim 1, wherein the composition comprises at least 10 g/L of a carbon source or a mixture of carbon sources.

49. The composition according to claim 1, wherein the composition comprises at least 50 g/L of a carbon source or a mixture of carbon sources.

50. The composition according to claim 1, wherein the oleaginous microbes are R. toruloides, wherein the composition comprises a carbon source, and wherein the concentration of the carbon source in the composition yields a higher lipid titer from the species R. toruloides as compared to a control composition with the species Y. lipolytica or L. starkeyi.

51. An oleaginous microbial fermentation broth composition, comprising:
a) a feedstock comprising at least 10 μM concentration of at least one oleaginous microbial inhibitor;
b) at least 10 g/L glycerol;
c) at least 0.5 grams (g) dry cell weight (DCW) per liter (L) oleaginous microbe titer; and
d) at least 0.2 g lipid per g DCW lipid content.

52. A method of producing an oleaginous microbial fermentation broth composition, comprising:
a) growing an oleaginous microbe on a feedstock comprising at least 10 μM concentration of at least one oleaginous microbial inhibitor,
wherein said method results in a microbially produced lipid content of at least 0.2 g lipid/g DCW.

53. The method according to claim 52, wherein the oleaginous microbial inhibitor is an acid.

54. The method according to claim 52, wherein the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.

55. The method according to claim 52, wherein the oleaginous microbial inhibitor is an aldehyde.

56. The method according to claim 52, wherein the oleaginous microbial inhibitor is 4-hydroxybenzaldehyde, furfural, or 5-hydroxymethyl-2-furaldehyde.

57. The method according to claim 52, wherein the oleaginous microbial inhibitor is an ester.

58. The method according to claim 52, wherein the oleaginous microbial inhibitor is propamocarb.

59. The method according to claim 52, wherein the oleaginous microbial inhibitor is a sugar alcohol.

60. The method according to claim 52, wherein the oleaginous microbial inhibitor is

61. The method according to claim 52, wherein the feedstock comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

62. The method according to claim 52, wherein the feedstock comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

63. The method according to claim 52, wherein the feedstock comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

64. The method according to claim 52, wherein the feedstock comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

65. The method according to claim 52, wherein the feedstock comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

66. The method according to claim 52, wherein the feedstock comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl furaldehyde, propamocarb, and xylitol.

67. The method according to claim 52, wherein the feedstock comprises at least one of the following oleaginous microbial inhibitors: 5-aminolevulinic acid, 4-hydroxybenzaldehyde, and 5-hydroxymethyl-2-furaldehyde.

68. The method according to claim 52, wherein the feedstock comprises at least two or three of the following oleaginous microbial inhibitors: 5-aminolevulinic acid, 4-hydroxybenzaldehyde, and 5-hydroxymethyl-2-furaldehyde.

69. The method according to claim 52, wherein the feedstock comprises each of the following oleaginous microbial inhibitors: 5-aminolevulinic acid, 4-hydroxybenzaldehyde, and 5-hydroxymethyl-2-furaldehyde.

70. The method according to claim 52, wherein the feedstock comprises 4-hydroxybenzaldehyde.

71. The method according to claim 52, wherein the feedstock comprises at least one of the following: at least 5 mg calcium per 100 g feedstock; at least 0.4 mg iron per 100 g feedstock; at least 100 mg potassium per 100 g feedstock; and at least 10 mg sodium per 100 g feedstock.

72. The method according to claim 52, wherein the feedstock comprises each of the following: at least 5 mg calcium per 100 g feedstock; at least 0.4 mg iron per 100 g feedstock; at least 100 mg potassium per 100 g feedstock; and at least 10 mg sodium per 100 g feedstock.

73. The method according to claim 52, wherein the oleaginous microbes are oleaginous yeast.

74. The method according to claim 52, wherein the oleaginous microbes are oleaginous yeast of the genus Rhodosporidium, Yarrowia, or Lipomyces.

75. The method according to claim 52, wherein the oleaginous microbes are oleaginous yeast of the genus Rhodosporidium.

76. The method according to claim 52, wherein the oleaginous microbes are oleaginous yeast of the species Rhodosporidium toruloides, Yarrowia lipolytica, or Lipomyces starkeyi.

77. The method according to claim 52, wherein the oleaginous microbes are oleaginous yeast of the species Rhodosporidium toruloides.

78. The method according to claim 52, wherein the method results in a DCW of at least 5.0 g/L.

79. The method according to claim 52, wherein the method results in a DCW of at least 10.0 g/L.

80. The method according to claim 52, wherein the method results in a DCW of at least 50.0 g/L.

81. The method according to claim 52, wherein the feedstock is a yeast fermentation waste product.

82. The method according to claim 52, wherein the feedstock is obtained from a yeast-based bioethanol production waste stream.

83. The method according to claim 52, wherein the feedstock is not obtained from food waste or hydrolysate from agricultural waste.

84. The method according to claim 52, wherein the feedstock is not obtained from a lignocellulosic biomass hydrolysate.

85. The method according to claim 52, wherein the method results in a lipid titer of at least 5 g/L.

86. The method according to claim 52, wherein the method results in a lipid titer of at least 10 g/L.

87. The method according to claim 52, wherein the method results in a lipid titer of at least 25 g/L.

88. The method according to claim 52, wherein the feedstock comprises a concentration of 4-hydroxybenzaldehyde that induces a higher lipid titer compared to the feedstock without 4-hydroxybenzaldehyde.

89. The method according to claim 52, wherein the lipid content is at least 0.3 g lipid/g DCW.

90. The method according to claim 52, wherein the lipid content is at least 0.5 g lipid/g DCW.

91. The method according to claim 52, wherein the feedstock is not pre-treated.

92. The method according to claim 52, wherein the feedstock is not detoxified, hydrolyzed, or treated with activated charcoal.

93. The method according to claim 52, wherein the feedstock is not pre-treated with physical, physico-chemical, chemical, or biological means.

94. The method according to claim 52, wherein the feedstock comprises a carbon source.

95. The method according to claim 52, wherein the feedstock is a yeast fermentation waste product, and wherein the composition comprises a carbon source not originally present in the feedstock.

96. The method according to claim 52, wherein the composition comprises a C3-C12 carbon source.

97. The method according to claim 52, wherein the composition comprises a carbon source selected from arabinose, glucose, glycerol, sucrose, and xylose, and any combination thereof.

98. The method according to claim 52, wherein the composition comprises a carbon source, and wherein the carbon source is glycerol.

99. The method according to claim 52, wherein the composition comprises at least 10 g/L of a carbon source or a mixture of carbon sources.

100. The method according to claim 52, wherein the composition comprises at least 50 g/L of a carbon source or a mixture of carbon sources.

101. The method according to claim 52, wherein the oleaginous microbes are R. toruloides, wherein the composition comprises a carbon source, and wherein the concentration of the carbon source in the composition yields a higher lipid titer from the species R. toruloides as compared to a control composition with the species Y. lipolytica or L. starkeyi.

102. A method of producing microbial lipids from oleaginous microbes, comprising:
a) providing a feedstock comprising at least 10 μM concentration of at least one oleaginous microbial inhibitor; and
b) growing the oleaginous microbes on said feedstock, thereby producing microbial lipids.

103. The method according to claim 102, wherein the oleaginous microbial inhibitor is an acid.

104. The method according to claim 102, wherein the oleaginous microbial inhibitor is an acid selected from the following list of acids: 5-aminolevulinic acid, mevalonic acid lactone, pyroglutamic acid, p-hydroxyphenyllactic acid, salicylic acid, alpha-hydroxyisocaproic acid, succinic acid-2,2,3,3-d4, and citric acid.

105. The method according to claim 102, wherein the oleaginous microbial inhibitor is an aldehyde.

106. The method according to claim 102, wherein the oleaginous microbial inhibitor is 4-hydroxybenzaldehyde, furfural, or 5-hydroxymethyl-2-furaldehyde.

107. The method according to claim 102, wherein the oleaginous microbial inhibitor is an ester.

108. The method according to claim 102, wherein the oleaginous microbial inhibitor is propamocarb.

109. The method according to claim 102, wherein the oleaginous microbial inhibitor is a sugar alcohol.

110. The method according to claim 102, wherein the oleaginous microbial inhibitor is xylitol.

111. The method according to claim 102, wherein the feedstock comprises at least one of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

112. The method according to claim 102, wherein the feedstock comprises at least two, three, four, five, six, seven, eight, nine, ten, eleven, or twelve of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

113. The method according to claim 102, wherein the feedstock comprises each of the following oleaginous microbial inhibitors: 5-aminolevulinic acid, mevalonic acid, pyroglutamic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

114. The method according to claim 102, wherein the feedstock comprises at least one of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

115. The method according to claim 102, wherein the feedstock comprises at least two, three, four, five, six, seven, eight, nine, or ten of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl-2-furaldehyde, propamocarb, and xylitol.

116. The method according to claim 102, wherein the feedstock comprises each of the following oleaginous microbial inhibitors: 5-aminolevulinic acid, p-hydroxyphenyllactic acid, salicylic acid, alpha-hydroxyisocaproic acid, succinic acid-2,2,3,3-d4, citric acid, 4-hydroxybenzaldehyde, furfural, 5-hydroxymethyl furaldehyde, propamocarb, and xylitol.

117. The method according to claim 102, wherein the feedstock comprises at least one of the following oleaginous microbial inhibitors: 5-aminolevulinic acid, 4-hydroxybenzaldehyde, and 5-hydroxymethyl-2-furaldehyde.

118. The method according to claim 102, wherein the feedstock comprises at least two or three of the following oleaginous microbial inhibitors: 5-aminolevulinic acid, 4-hydroxybenzaldehyde, and 5-hydroxymethyl-2-furaldehyde.

119. The method according to claim 102, wherein the feedstock comprises each of the following oleaginous microbial inhibitors: 5-aminolevulinic acid, 4-hydroxybenzaldehyde, and 5-hydroxymethyl-2-furaldehyde.

120. The method according to claim 102, wherein the feedstock comprises 4-hydroxybenzaldehyde.

121. The method according to claim 102, wherein the feedstock comprises at least one of the following: at least 5 mg calcium per 100 g feedstock; at least 0.4 mg iron per 100 g feedstock; at least 100 mg potassium per 100 g feedstock; and at least 10 mg sodium per 100 g feedstock.

122. The method according to claim 102, wherein the feedstock comprises each of the following: at least 5 mg calcium per 100 g feedstock; at least 0.4 mg iron per 100 g feedstock; at least 100 mg potassium per 100 g feedstock; and at least 10 mg sodium per 100 g feedstock.

123. The method according to claim 102, wherein the oleaginous microbes are oleaginous yeast.

124. The method according to claim 102, wherein the oleaginous microbes are oleaginous yeast of the genus Rhodosporidium, Yarrowia, or Lipomyces.

125. The method according to claim 102, wherein the oleaginous microbes are oleaginous yeast of the genus Rhodosporidium.

126. The method according to claim 102, wherein the oleaginous microbes are oleaginous yeast of the species Rhodosporidium toruloides, Yarrowia lipolytica, or Lipomyces starkeyi.

127. The method according to claim 102, wherein the oleaginous microbes are oleaginous yeast of the species Rhodosporidium toruloides.

128. The method according to claim 102, wherein the method results in a DCW of at least 5.0 g/L.

129. The method according to claim 102, wherein the method results in a DCW of at least 10.0 g/L.

130. The method according to claim 102, wherein the method results in a DCW of at least 50.0 g/L.

131. The method according to claim 102, wherein the feedstock is a yeast fermentation waste product.

132. The method according to claim 102, wherein the feedstock is obtained from a yeast-based bioethanol production waste stream.

133. The method according to claim 102, wherein the feedstock is not obtained from food waste or hydrolysate from agricultural waste.

134. The method according to claim 102, wherein the feedstock is not obtained from a lignocellulosic biomass hydrolysate.

135. The method according to claim 102, wherein the method results in a lipid titer of at least 5 g/L.

136. The method according to claim 102, wherein the method results in a lipid titer of at least 10 g/L.

137. The method according to claim 102, wherein the method results in a lipid titer of at least 25 g/L.

138. The method according to claim 102, wherein the feedstock comprises a concentration of 4-hydroxybenzaldehyde that induces a higher lipid titer compared to the feedstock without 4-hydroxybenzaldehyde.

139. The method according to claim 102, wherein the method results in a lipid content of at least 0.3 g lipid/g DCW.

140. The method according to claim 102, wherein the method results in a lipid content of at least 0.5 g lipid/g DCW.

141. The method according to claim 102, wherein the feedstock is not pre-treated.

142. The method according to claim 102, wherein the feedstock is not detoxified, hydrolyzed, or treated with activated charcoal.

143. The method according to claim 102, wherein the feedstock is not pre-treated with physical, physico-chemical, chemical, or biological means.

144. The method according to claim 102, wherein the feedstock comprises a carbon source.

145. The method according to claim 102, wherein the feedstock is a yeast fermentation waste product, and wherein the composition comprises a carbon source not originally present in the feedstock.

146. The method according to claim 102, wherein the feedstock comprises a C3-C12 carbon source.

147. The method according to claim 102, wherein the feedstock comprises a carbon source selected from arabinose, glucose, glycerol, sucrose, and xylose, and any combination thereof.

148. The method according to claim 102, wherein the feedstock comprises a carbon source, and wherein the carbon source is glycerol.

149. The method according to claim 102, wherein the feedstock comprises at least 10 g/L of a carbon source or a mixture of carbon sources.

150. The method according to claim 102, wherein the feedstock comprises at least 50 g/L of a carbon source or a mixture of carbon sources.

151. The method according to claim 102, wherein the oleaginous microbes are R. toruloides, wherein the feedstock comprises a carbon source, and wherein the concentration of the carbon source in the feedstock yields a higher lipid titer from the species R. toruloides as compared to a control feedstock with the species Y. lipolytica or L. starkeyi.
